(19) United States
(12) Patent Application Publication
(10) Pub. No.: US 2017/0312018 A1
(43) Pub. Date: Nov. 2, 2017

(54) ELECTROSURGICAL INSTRUMENT WITH CONDUCTIVE GAP SETTING MEMBER AND INSULATIVE TISSUE ENGAGING MEMBER HAVING VARIABLE DIMENSIONS AND STIFFNESS

(71) Applicant: Ethicon Endo-Surgery, LLC, Guaynabo, PR (US)

(72) Inventors: Gregory A. Trees, Loveland, OH (US); Megan A. Broderick, West Chester, OH (US); Paul T. Franer, Cincinnati, OH (US); Robert J. Sanzone, Cincinnati, OH (US); Frederick E. Shelton, IV, Hillsboro, OH (US); Mark A. Davison, Maineville, OH (US); David C. Yates, West Chester, OH (US)

(21) Appl. No.: 15/142,598

(22) Filed: Apr. 29, 2016

(51) Int. Cl. A61B 18/14; A61B 18/00; A61B 17/00

(52) U.S. Cl. CPC A61B 18/1447; A61B 2018/1455; A61B 2018/0063; A61B 2017/00526; A61B 2018/00595; A61B 2018/00589; A61B 2018/00601

(57) ABSTRACT

An end effector has a first jaw member that includes a first electrode, and second jaw member that includes a second electrode, an electrically conductive member located at the distal end of either the first jaw member or the second jaw member, and an electrically insulative member located either on the first jaw member or the second jaw member. The electrically conductive member is configured to define a distance between the first and second electrodes along the length of the first and second electrodes, the electrically conductive member having a first stiffness. The electrically insulative member is located either on the first jaw member or the second jaw member and is sized and configured to engage tissue. The electrically conductive member has a first stiffness and the electrically insulative member has a second stiffness. The first stiffness is greater than the first stiffness.

### RELATED APPLICATIONS

This application is related to the following commonly owned patent applications referenced under:

Attorney Docket No. END7918USNP/160047 entitled ELECTROSURGICAL INSTRUMENT WITH ELECTRICALLY CONDUCTIVE GAP SETTING MEMBER AND ELECTRICALLY INSULATIVE TISSUE ENGAGING MEMBERS;

Attorney Docket No. END7920USNP/160049 entitled ELECTROSURGICAL INSTRUMENT WITH ELECTRICALLY CONDUCTIVE GAP SETTING AND TISSUE ENGAGING MEMBERS;

Attorney Docket No. END7921USNP/160059 entitled JAW STRUCTURE WITH DISTAL POST FOR ELECTROSURGICAL INSTRUMENTS;

Attorney Docket No. END7922USNP/160060 entitled NON-LINEAR JAW GAP FOR ELECTROSURGICAL INSTRUMENTS; and

Attorney Docket No. END7923USNP/160061 entitled JAW STRUCTURE WITH DISTAL CLOSURE FOR ELECTROSURGICAL INSTRUMENTS; each of which is incorporated herein by reference in its entirety.

### TECHNICAL FIELD

The present disclosure is related generally to medical devices having various mechanisms for grasping and sealing tissue. In particular, the present disclosure is related to medical devices having an electrically conductive gap setting member configured to define a gap between electrodes of an electrosurgical instrument.

### BACKGROUND

Electrosurgical devices may be used in many surgical operations. Electrosurgical devices may apply electrical energy to tissue in order to treat tissue. An electrosurgical device may comprise an instrument having a distally mounted end effector comprising one or more electrodes. The end effector can be positioned against tissue such that electrical current may be introduced into the tissue. Electrosurgical devices can be configured for monopolar or bipolar operation. During monopolar operation, current may be introduced into the tissue by an active (or source) electrode on the end effector and returned through a return electrode. The return electrode may be a grounding pad and separately located on a patient's body. During bipolar operation, current may be introduced into and returned from the tissue by the active and return electrodes, respectively, of the end effector.

The end effector may include two or more jaw members. At least one of the jaw members may have at least one electrode. At least one jaw may be moveable from a position spaced apart from the opposing jaw for receiving tissues to a position in which the space between the jaw members is less than that of the first position. This movement of the moveable jaw may compress the tissue held between. Heat generated by the current flow through the tissue in combination with the compression achieved by the jaw's movement may form hemostatic seals within the tissue and/or between tissues and, thus, may be particularly useful for sealing blood vessels, for example. The end effector may comprise a cutting member. The cutting member may be movable relative to the tissue and the electrodes to transect the tissue.

Electrosurgical devices also may include mechanisms to clamp tissue together, such as a stapling device, and/or mechanisms to sever tissue, such as a tissue knife. An electrosurgical device may include a shaft for placing the end effector proximate to tissue undergoing treatment. The shaft may be straight or curved, bendable or non-bendable. In an electrosurgical device including a straight and bendable shaft, the shaft may have one or more articulation joints to permit controlled bending of the shaft. Such joints may permit a user of the electrosurgical device to place the end effector in contact with tissue at an angle to the shaft when the tissue being treated is not readily accessible using an electrosurgical device having a straight, non-bending shaft.

### SUMMARY

In one aspect, an end effector comprises a first jaw member comprising a first electrode, a distal end, and a proximal end. The end effector also comprises a second jaw member comprising a second electrode, wherein at least one of the first and second jaw members is movable relative to the other between an open position and a closed position, the second jaw member comprising a distal end and a proximal end. In addition, the end effector comprises an electrically conductive member located at the distal end of the first jaw member, the electrically conductive member is sized and configured to define a minimum distance between the first and second electrodes, the electrically conductive member having a first stiffness. Furthermore, the end effector comprises an electrically insulative member located on either one of the first jaw member or the second jaw member, the electrically insulative member sized and is sized and configured to engage tissue, the electrically insulative member having a second stiffness, wherein the first stiffness is greater than the second stiffness.

In one aspect, an electrosurgical device comprises a handle assembly, an end effector operably coupled to the handle assembly. The end effector also comprises a second jaw member comprising a second electrode, wherein at least one of the first and second jaw members is movable relative to the other between an open position and a closed position, the second jaw member comprising a distal end and a proximal end. In addition, the end effector comprises an electrically conductive member located at the distal end of the first jaw member, the electrically conductive member is sized and configured to define a minimum distance between the first and second electrodes, the electrically conductive member having a first stiffness. Furthermore, the end effector comprises an electrically insulative member located on either one of the first jaw member or the second jaw member, the electrically insulative member sized and is sized and configured to engage tissue, the electrically insulative member having a second stiffness, wherein the first stiffness is greater than the second stiffness. The electrosurgical instrument also comprises a connecting member configured to connect the handle assembly and the end effector.

In one aspect, an electrosurgical system comprises an electrosurgical energy generator; an electrosurgical device comprising a handle assembly, an end effector operably coupled to handle assembly, and a connecting member configured to connect the handle assembly and the end effector. The end effector also comprises a second jaw member comprising a second electrode, wherein at least one of the first and second jaw members is movable relative to the other between an open position and a closed position, the second jaw member comprising a distal end and a proximal end. In addition, the end effector comprises an electrically conductive member located at the distal end of the first jaw member, the electrically conductive member is sized and configured to define a minimum distance between the first and second electrodes, the electrically conductive member having a first stiffness. Furthermore, the end effector comprises an electrically insulative member located on either one of the first jaw member or the second jaw member, the electrically insulative member sized and is sized and configured to engage tissue, the electrically insulative member having a second stiffness, wherein the first stiffness is greater than the second stiffness.

In one aspect, a method of manufacturing an end effector comprising a first jaw member, a second jaw member, and an electrically insulative member assembly, the method comprises providing a position setting material in an inner portion of the first jaw member; inserting into the inner portion of the first jaw member the electrically insulative member assembly; and providing a position setting member between the first and second jaw members, the position setting member having non-uniform thickness.

### BRIEF DESCRIPTION OF THE DRAWINGS

The features of the various aspects are set forth with particularity in the appended claims. The various aspects, both as to organization and methods of operation, together with advantages thereof, may, however best be understood by reference to the following description, taken in conjunction with the accompanying drawings as follows:

FIG. 1A shows a surgical instrument in electrical communication with an energy source, according to one aspect of the present disclosure.

FIG. 1B is a detailed view of the end effector of the surgical instrument shown in FIG. 1A, according to one aspect of the present disclosure.

FIG. 2A and FIG. 2B show two types of electrically insulative members, according to one aspect of the present disclosure.

FIG. 3 shows a plan view of one aspect of a jaw member comprising a distal electrically conductive gap setting member and electrically insulative tissue engaging members having variable sizes along the jaw member, according to one aspect of the present disclosure.

FIG. 4 shows a plan view of one aspect of a jaw member comprising a distal electrically conductive gap setting member and electrically insulative tissue engaging members having variable sizes and/or shapes along the jaw member, according to one aspect of the present disclosure.

FIG. 5 shows a side elevational view of one aspect of an end effector comprising a distal electrically conductive gap setting member and electrically insulative tissue engaging members, according to one aspect of the present disclosure.

FIG. 6 shows a side elevational view of one aspect of an end effector comprising a distal electrically conductive gap setting member and electrically insulative tissue engaging members non-uniform stiffness, according to one aspect of the present disclosure.

FIG. 7 shows a side elevational view of one aspect of an end effector comprising a distal electrically conductive gap setting member and compliant electrically insulative tissue engaging members, according to one aspect of the present disclosure.

FIG. 8A and FIG. 8B show one example process of manufacturing an end effector comprising a distal electrically conductive gap setting member and electrically insulative tissue engaging members assembly using a shim, according to one aspect of the present disclosure.

FIG. 9 shows a side elevational view of one aspect of an end effector comprising a distal electrically conductive gap setting member, electrically insulative tissue engaging members, cambered electrodes, and electrically insulative members contacting the opposing electrode, according to one aspect of the present disclosure.

FIG. 10 shows a side elevational view of one aspect of an end effector comprising a distal electrically conductive gap setting member, electrically insulative tissue engaging members, cambered electrodes, and electrically insulative members not contacting the opposing electrode, according to one aspect of the present disclosure.

FIG. 11 shows a side elevational view of one aspect of an end effector comprising a distal electrically conductive gap setting member, electrically insulative tissue engaging members, and a compliant upper jaw member, according to one aspect of the present disclosure.

FIG. 12 shows a cross-sectional view of an end effector comprising a distal electrically conductive gap setting member and electrically insulative tissue engaging members provided on a stepped or terrace electrode, according to one aspect of the present disclosure.

FIG. 13 shows a cross-sectional view of one aspect of an end effector comprising a distal electrically conductive gap setting member and electrically insulative tissue engaging members on one jaw member and buried in recesses on another other jaw member, according to one aspect of the present disclosure.

FIG. 14 shows a cross-sectional view of one aspect of an end effector comprising a distal electrically conductive gap setting member and compliant electrically insulative tissue engaging members deforming uniformly under the same pressure, according to one aspect of the present disclosure.

FIG. 15 shows a plan view of one aspect of a jaw member comprising a distal electrically conductive gap setting member and electrically insulative tissue engaging members evenly positioned along a width of the jaw member, according to one aspect of the present disclosure.

FIG. 16 shows a XVI-XVI sectional view of one aspect of an end effector with the jaw of FIG. 15, according to one aspect of the present disclosure.

FIG. 17 shows a plan view of one aspect of a jaw member comprising a distal electrically conductive gap setting member and electrically insulative tissue engaging members unevenly positioned along a width of the jaw member, according to one aspect of the present disclosure.

FIG. 18 shows a XVIII-XVIII sectional view of one aspect of an end effector with the jaw member of FIG. 17, according to one aspect of the present disclosure.

FIG. 19 is a side elevational view of the end effector of FIG. 5, according to one aspect of the present disclosure.

FIG. 20 shows a perspective view of the lower jaw member of FIG. 5, according to one aspect of the present disclosure.

FIG. 21 shows a side elevational view of the lower jaw member of FIG. 20, according to one aspect of the present disclosure.

FIG. 22 shows a detail view of the distal end of the lower jaw member of FIG. 21, according to one aspect of the present disclosure.

FIG. 23 shows a plan view of the lower jaw member of FIG. 20, according to one aspect of the present disclosure.

FIG. 24 shows a detail plan view of the distal end of the lower jaw member of FIG. 23, according to one aspect of the present disclosure.

### DETAILED DESCRIPTION

The following description of certain examples of the technology should not be used to limit its scope. Other examples, features, aspects, aspects, and advantages of the technology will become apparent to those skilled in the art from the following description, which is, by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, aspects, examples, etc. described herein may be combined with any one or more of the other teachings, expressions, aspects, examples, etc. that are described herein. The following described teachings, expressions, aspects, examples, etc. should, therefore, not be viewed in isolation relative to each other. Various suitable ways in which the teachings herein may be combined will be readily apparent to those of ordinary skill in the art in view of the teachings herein. Such modifications and variations are intended to be included within the scope of the claims.

Also, in the following description, it is to be understood that terms such as front, back, inside, outside, upper, lower, top, bottom and the like are words of convenience and are not to be construed as limiting terms. Terminology used herein is not meant to be limiting insofar as devices described herein, or portions thereof, may be attached or utilized in other orientations. The various aspects will be described in more detail with reference to the drawings. Throughout this disclosure, the term “proximal” is used to describe the side of a component, e.g., a shaft, a handle assembly, etc., closer to a user operating the surgical instrument, e.g., a surgeon, and the term “distal” is used to describe the side of the component farther from the user operating the surgical instrument.

Aspects of the present disclosure are presented for a single electrosurgical device configured for grasping tissue and performing sealing procedures using electrical and/or other energy. An end effector of the electrosurgical device may include multiple members arranged in various configurations to collectively perform the aforementioned functions. As used herein, an end effector may be referred to as a jaw assembly or clamp jaw assembly comprising an upper jaw member and a lower jaw member where at least one of the upper jaw member and the lower jaw member may be movable relative to the other. Each of the jaw members may be adapted to connect to an electrosurgical energy source. Each jaw member may incorporate an electrode. The electrode may be a positive or negative electrode. In a bipolar electrosurgical device, the electrodes may be adapted for connection to the opposite terminals of the electrosurgical energy source, such as a bipolar radio frequency (RF) generator, so as to generate a current flow therebetween. An electrosurgical energy may be selectively communicated through tissue held between the jaw members to effect a tissue seal and/or treatment. Tissue may be coagulated from the current flowing between the opposite polarity electrodes on each jaw member.

At least one jaw member may include a knife channel defined therein configured to reciprocate a knife therealong for severing tissue held between the jaw members. The knife channel may be an extended slot in the jaw member. The knife may be provided within a recess associated with the at least one jaw member. The electrosurgical device may have both coagulation and cutting functions. This may eliminate or reduce instrument interchange during a surgery. Cutting may be achieved using mechanical force alone or a combination of mechanical force and the electrosurgical energy. The electrosurgical energy may be selectively used for coagulation and/or cutting. The knife may be made from an electrically conductive material adapted to connect to the electrosurgical source, and selectively activatable to separate tissue disposed between the jaw members. The knife may be spring biased such that once tissue is severed, the knife may automatically return to an unengaged position within the knife channel or a retracted position in the recess.

In some aspects, the jaw members may be movable relative to each other. During operation of the electrosurgical device, at least one of the jaw members may move from a first, open position where the jaw members can be disposed around a mass of tissue, to a second, closed position where the jaw members grasp the tissue. The jaw members therefore may move through a graspers-like range of motion, similar to that of conventional pliers. In the second position, current flows between the jaw members to achieve hemostasis of the tissue captured therebetween. The jaw members may be configured to have a relatively thick proximal portion to resist bending. At least one of the jaw members may have a three-dimensional configuration with a D-shaped cross-sectional. The three-dimensional configuration with the D-shaped cross-sectional may resist bending. A lock mechanism may be included to lock the jaw members in the closed position. The lock mechanism may set the clamp pressure between the jaw members. At least one electrically conductive gap setting member may be provided between the jaw members to establish a desired gap between electrodes in bipolar electrosurgical devices.

The electrosurgical device may incorporate components to set a gap between the jaws of the end effector, grasp a tissue via the end effector, deliver energy to the tissue via one or more electrodes, and cut the tissue via a dissecting device such as a tissue knife. The structural capabilities of any aspect of an electrosurgical device may be designed for use in one or more of a variety of surgical procedures. In some surgical procedures, the treated tissue may be readily accessible to an end effector affixed to a relatively straight and unbendable shaft. In some alternative surgical procedures, the tissue may not be readily accessible to the end effector on such a shaft. In such procedures, the electrosurgical device may incorporate a shaft designed to bend so that the end effector may contact the tissue requiring treatment. In such a device, the shaft may include one or more articulated joints that may permit the shaft to bend under control by the user. A sliding knife may include a feature to provide actuating force to the sliding knife. A knife actuator may be operably coupled to the shaft for selectively reciprocating the knife through the knife channel.

A front portion assembly may be designed for a specific surgical procedure, while a reusable handle assembly, configured to releasably attach to a front portion assembly, may be designed to provide control of surgical functions common to each front portion assembly, such as tissue grasping, cauterizing, and cutting. Consequently, the number and types of devices required for surgeries can be reduced. The reusable handle assembly may be designed to automate common functions of the electrosurgical device. Device intelligence may be provided by a controller located in the reusable handle assembly that is configured to receive information from a front portion assembly. Such information may include data regarding the type and use of the front portion assembly. Alternatively, information may include data indicative of the position and/or activation of control components (such as buttons or slides that can be manipulated) that may indicate what system functions should be activated and in what manner.

In some non-limiting examples, the controller may supply the RF current when the energy activation control is placed in an activating position by the user. In some alternative non-limiting examples, the controller may supply the RF current for a predetermined period of time once the energy activation control is placed in an activing position. In yet another non-limiting example, the controller may receive data related to the position of the jaws and prevent the RF current from being supplied to the to the one or more tissue cauterization power contacts if the jaws are not in a closed position.

In some aspects, any of the mentioned examples also may be configured to articulate along at least one axis through various means, including, for example, a series of joints, one or more hinges or flexure bearings, and one or more cam or pulley systems. Other features may include cameras or lights coupled to one or more of the members of the end effector, and various energy options for the surgical device.

The electrosurgical device can be configured to source energy in various forms including, without limitation, electrical energy, monopolar and/or bipolar RF energy, microwave energy, reversible and/or irreversible electroporation energy, and/or ultrasonic energy, heat energy, or any combination thereof, to the tissue of a patient either independently or simultaneously. The energy can be transmitted to the electrosurgical device by a power source in electrical communication with the electrosurgical device. The power source may be a generator. The power source may be connected to the electrosurgical device via a suitable transmission medium such as a cable. The power source may be separate from the electrosurgical device or may be made integrally with the electrosurgical device to form a unitary electrosurgical system. In one non-limiting example, the power source may include one or more batteries located within a portion of the electrosurgical device. It may be understood that the power source may source energy for use on the tissue of the patient as well as for any other electrical use by other devices, including, without limitation, lights, sensors, communication systems, indicators, and displays, which operate in relation to and/or with the electrosurgical device to form an electrosurgical system.

The electrosurgical device may be configured to source electrical energy in the form of RF energy. The electrosurgical device can transmit the RF energy through tissue compressed between two or more jaws. Such RF energy may cause ionic agitation in the tissue, in effect producing resistive heating, and thereby increasing the temperature of the tissue. Increased temperature of the tissue may lead to tissue cauterization. In some surgical procedures, RF energy may be useful for removing, shrinking, or sculpting soft tissue while simultaneously sealing blood vessels. RF energy may work particularly well on connective tissue, which is primarily composed of collagen and shrinks when contacted by heat. Because a sharp boundary may be created between the affected tissue and the surrounding tissue, surgeons can operate with a high level of precision and control, without sacrificing untargeted adjacent tissue.

The RF energy may be in a frequency range described in EN 60601-2-2:2009+A11:2011, Definition 201.3.218—HIGH FREQUENCY. For example, the frequency in monopolar RF applications may be typically restricted to less than 5 MHz. However, in bipolar RF applications, the frequency can be almost anything. Frequencies above 200 kHz can be typically used for monopolar applications in order to avoid the unwanted stimulation of nerves and muscles that would result from the use of low frequency current. Lower frequencies may be used for bipolar applications if the risk analysis shows the possibility of neuromuscular stimulation has been mitigated to an acceptable level. Normally, frequencies above 5 MHz are not used in order to minimize the problems associated with high frequency leakage currents. Higher frequencies may, however, be used in the case of bipolar applications. It is generally recognized that 10 mA is the lower threshold of thermal effects on tissue.

As discussed above, the electrosurgical device may be used in conjunction with a generator. The generator may be an electrosurgical generator characterized by a fixed internal impedance and fixed operating frequency that deliver maximum power to an external load (e.g., tissue) having an electrical impedance in the range of about 50 ohms to 150 ohms. In this type of bipolar electrosurgical generator, the applied voltage may increase monotonically as the load impedance increases toward the maximum “open circuit” voltage as the load impedance increases to levels of tens of thousands of ohms or more. In addition, the electrosurgical device may be used with a bipolar electrosurgical generator having a fixed operating frequency and an output voltage that may be substantially constant over a range of load impedances of tens of ohms to tens of thousands of ohms including “open circuit” conditions. The electrosurgical device may be advantageously used with a bipolar electrosurgical generator of either a variable voltage design or substantially constant voltage design in which the applied voltage may be interrupted when the delivered current decreases below a predetermined level. Such bipolar generators may be referred to as automatic generators in that they may sense the completion of the coagulation process and terminate the application of voltage, often accompanied by an audible indication in the form of a cessation of a “voltage application” tone or the annunciation of a unique “coagulation complete” tone. Further, the electrosurgical device may be used with an electrosurgical generator whose operating frequency may vary with the load impedance as a means to modulate the applied voltage with changes in load impedance.

Various aspects of electrosurgical devices use therapeutic and/or sub-therapeutic electrical energy to treat tissue. Some aspects may be utilized in robotic applications. Some aspects may be adapted for use in a hand operated manner. In one non-limiting example, an electrosurgical device may include a proximal handle, a distal working end or end effector, and an introducer or elongated shaft disposed in-between.

In some non-limiting medical procedures, the electrosurgical device may be used to weld or seal vessels prior to tissue resection. Such vessels also may be removed as part of procedures to resect other tissue such as cysts, tumors, or infected materials. Blood vessel sealing may reduce bleeding, thereby decreasing potential harmful effects during a resection procedure. In such procedures, vessels may be cut at the cauterization location. It may be understood that complete sealing may be required at the site of the cut to prevent bleeding. It is therefore useful to have an electrosurgical device that may be prevented from cutting a vessel until complete sealing is assured.

To properly seal vessels, two mechanical parameters that affect thickness of the sealed vessel may be accurately controlled: the pressure applied to the vessel and the gap between the electrodes. Proper sealing may require that sufficient pressure is placed on the vessel to assure that the vessel walls are proximate to each other and no intervening gap remains therebetween. The vessel may be compressed to a pressure within a predetermined range. A typical range of appropriate pressures may be between 30 and 250 pounds per square inch (psi). In addition, proper sealing may require that sufficient power is provided to assure that the vessel walls receive sufficient heat to weld the walls together. Thus, both tissue compression and tissue cauterization may be required to form a proper seal. These can be achieved by the jaw members of the end effector. As mentioned above, the jaw members may grasp, compress, and deliver the energy to the tissue.

To effectively carry out hemostasis, the jaw members should efficiently conduct a proper current flow through the grasped tissue. When that current is insufficient, coagulation of the tissue or vessel may be compromised. When the current is excessive, correspondingly excessive heating may occur with a potential for the generation of damaging electrical arcing. Excessive heating may result in the phenomenon of tissue and blood coagulum sticking to the surface of the jaw members. This may result in increased electrical impedance between the electrodes of the device and the tissue that may subsequently be grasped for the purpose of treatment. Such sticking tissue may evoke a disruption of the coagulated surface, which in itself may compromise the intended hemostatic effect. The end effector may incorporate highly polished electrode surfaces for the purpose of reducing the extent of tissue sticking as well as to facilitate their cleaning when sticking does occur. When grasping tissue, the jaw members may come into mutual contact, causing a short circuit. For example, when a small tissue component is grasped between the jaw members and/or when the jaw members are compressed hard, the electrodes may be in contact with each other in the vicinity of the grasped tissue, causing short-circuiting. The jaw members may include insulative coatings that may be in contact in some geometry, but the insulative coatings may not prevent the short-circuiting.

Arcing may be a possibility as the jaw members closely approach each other. Arcing may happen when monopolar electrosurgical devices are used where the current flows completely through the patient. These high voltage electrical currents may arc from the small electrode to nearby, non-targeted vital structures or may follow erratic paths as they flow through the patient's body, thereby causing damage to tissues both near and at some distance from the electrode. Aberrant current arcs may cause deep tissue necrosis and inadvertent damage to adjacent tissue masses.

Arcing also may happen in a procedure performed by a bipolar electrosurgical device, for example, a “coagulative painting” procedure, where the side surfaces of the electrically active jaw members are drawn over the surface of membranous tissue such as the mesentery. Done properly, this action congeals the microvessels within such thin tissues. However, higher voltage settings on the generator applied across a thin layer of tissue to the other jaw member can cause arcing of the device. For some bipolar electrosurgical devices, microarcs between the electrodes may be normal during operation. However, these microarcs can attack the electrodes. If the electrodes, for example, contain some polymer material, these microarcs can draw out carbon from the polymer material, thus creating carbon tracks, sometimes referred to as “carbon arc tracking,” which then may lead to short-circuiting of the electrodes. Also, in general, in case of excessive voltage or sharp edges, a significant arc or a big arc may happen, and the generator may perceive the arc as short-circuiting. Short-circuiting due to either a big arc or carbon arc tracking can be very problematic. This calls for adjustment of the voltage or maintenance of the spacing between the two jaw members to avoid arcing the system. It may be desirable to adjust the spacing rather than changing the applied voltage because lowering the voltage may result in less than desirable tissue effects. Of course, it is also necessary for the surgeon to maintain space between the electrodes of the device to achieve the requisite performance.

In general, for bipolar electrosurgical devices, electrodes of opposite polarity should not contact each other during the application of energy. Shorting of the electrodes effectively shunts energy away from the tissue. Some shunting happens with arcing. It is known that Paschen's Law gives the breakdown voltage, which is the voltage necessary to start a discharge or electric arc between two electrodes in a gas as a function of pressure and gap length. The breakdown voltage of various gases between parallel metal plates as the gas pressure and gap distance were varied has been studied. It has been found that the voltage necessary to arc across the gap decreases as the pressure is reduced and then increased gradually, exceeding its original value. It has also been found that at normal pressure, the voltage needed to cause an arc reduces as the gap size is reduced but only to a point. As the gap is reduced further, the voltage required to cause an arc begins to rise and again exceeds its original value. For a given gas, the voltage is a function only of the product of the pressure and gap length. According to Paschen's Law, at higher pressures and gap lengths, the breakdown voltage is approximately proportional to the product of pressure and gap length. If a bipolar device allows shorting or arcing between the tissue treating electrodes, the effectiveness of the device may be diminished. In one aspect, present disclosure provides an electrically conductive gap setting member to prevent one electrode from contacting the opposed electrode of a bipolar electrosurgical device. In various aspects, the electrically conductive gap setting member may define a uniform or non-uniform gap along the length and/or the width of the jaw member(s) or tissue contacting area thereof.

According to various aspects, an end effector may include an electrically conductive gap setting member to ensure that the electrodes of the jaw members do not electrically contact each other within a range of the closing or opening motion of the jaw members. The electrically conductive gap setting member defines a gap between the upper and lower electrodes of the jaw members when the jaw members are at the closed position. The gap may be uniform or non-uniform along the length and/or width of the tissue contacting area of the jaw. The electrically conductive gap setting member may be dimensioned so that when the jaw members are in the closed position, the gap may be sufficient to prevent electrical shorting between the electrodes. The electrically conductive gap setting member may control the gap distance between opposing electrodes of the jaw members. The heights of the electrically conductive gap setting members are selected as the value to achieve a minimum spacing between the electrode surfaces driving a current path through the grasped tissue, which may be of a distance that does not exceed a value necessary to achieve effective coagulation while avoiding arcing and/or short-circuiting. Although the electrically conductive gap setting member is made of an electrically conductive material, the electrically conductive gap setting member is electrically isolated from the electrode connected to the positive terminal or pole of the energy source and may contact the electrode connected to the negative or ground terminal or pole of the energy source.

In various aspects, the electrically insulative tissue engaging member may comprise an insulative layer or coating. The insulative layer may have a thickness in the range of about 0.002″ to about 0.050″, more preferably about 0.003″ to about 0.007″. At thicknesses of about 0.001″ or less, the thickness of the insulative layer may be insufficient to prevent shorting of the electrodes. Insulative layer thicknesses above about 0.002″ and below about 0.050″ may cause adequate hemostasis. It has been observed, however, that the greater the minimum distance between the proximate current conducting portions of the opposing electrodes in the region of current flow through the tissue, the longer the current path through the tissue and the more difficult it may become to obtain the desired localized and intense heating to achieve adequate hemostasis. Insulative layer thicknesses above about 0.050″ may be too large for most practical applications using the ceramic insulative materials described.

In various aspects, an electrically conductive gap setting member may be provided between the jaw members. The electrically conductive gap setting member may be affixed on and/or integral to one jaw member and extend to the other jaw member. The electrically conductive gap setting member may protrude through the jaw member. The electrically conductive gap setting member may define a gap between the jaw members. The electrically conductive gap setting member may be electrically conductive. The electrically conductive gap setting member may be a pin. The pin may be metal. The gap setting member can be made of a material that is electrically conductive and also is stiff to resist deformation in response to an applied force. The material is stiff with a high tensile strength and is incompressible. The electrically conductive gap setting member can be made of an electrically conductive metal or metal alloy and preferably can be made of steel, such as medical grade stainless steel, for example. The electrically conductive gap setting member may not contact the electrically conductive surface or portion of any electrode, including the electrode which the electrically conductive gap setting member may be affixed on or protrude through and the opposite electrode. The electrically conductive gap setting member may be sized and configured to avoid short-circuiting between the opposing electrodes and/or ensure that the electrodes would not close enough to arc without the presence of tissue between the electrodes.

In various aspects, the gap between the jaw members or the electrodes may be about 0.002″ to about 0.02″, preferably about 0.003″ to about 0.012″, more preferably about 0.004″ to about 0.01″, even more preferably about 0.004″ to about 0.008″. The gap between the electrode and the upper of the electrically insulative tissue engaging member on the opposite electrode may be about 0 to about 0.005″, preferably about 0.001″ to about 0.005″, more preferably about 0.001″ to about 0.002″, more preferably about 0.001″. These gaps may be configured to provide desired sealing of vessels. As smaller distances between the electrodes are employed, for example, at values of about 0.001″ or about 0.002″, arcing may occur. For example, it has been found that as the height diminishes below about 0.005″, for example, to about 0.001″ or about 0.002″, isotonic saline fluid is encountered in the surgical field and the spacing between grasping surfaces, and an arc may form and evoke intense heating in its ionized pathway with resultant damage.

According to various aspects, an end effector may include an electrically insulative member between the jaw members. The electrically insulative member may be provided on at least one of the jaw members. Each jaw member may have a surface. The surface may be a tissue grasping surface. The surface may comprise an electrode. The surface of the upper jaw member may face the surface of the lower jaw member. The electrically insulative member may comprise at least one electrically insulative tissue engaging member. The at least one electrically insulative tissue engaging member is a protuberance in the form of a short cylindrical solid or hollow object, bump, hump, lump, ridge, bulge, knob, swelling peg, or buttonmade integral with or inserted into a jaw member and protruding through openings defined by an electrode of the jaw member. The electrically insulative tissue engaging members are configured to facilitate gripping or grasping tissue located between the jaw members and enhance manipulation of tissue during the operation of the electrosurgical device, such as the sealing process.

In some aspects where there may be more than one electrically insulative member, the more than one electrically insulative member may be provided on the same surface or on difference surfaces of the jaw members. In some aspects where at least one electrically insulative member may be provided on one surface of a jaw member, effective grasping of very thin tissue and small blood vessels may be provided. Manufacturing costs may be reduced as the at least one electrically insulative member need only be applied to one of the two jaw members. Because it is not required to have the electrically insulative member on both jaw members, it may not be required to precisely control the widths of more than one insulative member and the spacing therebetween to assure required registration between an upper and lower disposed array of electrically insulative members. This may reduce manufacturing costs. This may enhance manufacturability inasmuch as the requirement for precisely registering the insulative members at two grasping surfaces of the jaw members may be eliminated during final assembly.

In various aspects, at least one electrode may be made on at least one surface of the at least one jaw member. The electrically insulative tissue engaging member may protrude from an opening in the electrode. In some non-limiting examples, the opening in the electrode may be line-line same size as the member protruding from the opening. Therefore, the electrically insulative tissue engaging member may be a tight fit through the opening. In some other non-limiting examples, the opening in the electrode may be larger than the electrically insulative tissue engaging member and thereby form a donut around the electrically insulative tissue engaging member. When the opening is larger than the electrically insulative tissue engaging member, it may be easier for manufacturing since it may be easier to align the electrically insulative tissue engaging members if needed. The opening may have a diameter twice as large as a diameter of the electrically insulative tissue engaging member. In some aspects, the opening may have a size such that the space around the electrically insulative tissue engaging member may allow the electrically insulative tissue engaging member to move and/or deform. In any case, the opening, the electrically insulative tissue engaging member, and the space therebetween should have appropriate sizes and/or dimensions such that the electrosurgical device and its electrodes achieve the requisite performance.

In various aspects, the at least one electrically insulative tissue engaging member may have various shapes. The at least one electrically insulative tissue engaging member may have the shape of a cube, rectangular prism, triangular prism, octagonal prism, tetrahedron, square pyramid, cylinder, cone, sphere, or any other suitable shape. A upper surface of the at least one electrically insulative tissue engaging member may be round, square, rectangle, oval, or any other suitable shape. In some aspects where there is more than one electrically insulative tissue engaging member, the electrically insulative tissue engaging members may each have the same shape or different shapes with any combination of various shapes. In certain aspects, the top surface can be smooth or patterned.

In various aspects, there may be more than one electrically insulative tissue engaging member. The electrically insulative tissue engaging members may have different shapes and/or sizes. All or some of the electrically insulative tissue engaging members may change shapes and/or sizes along the length of the electrodes. The electrically insulative tissue engaging members may have increasing or decreasing sizes along the length of the electrodes. The electrically insulative tissue engaging members may change shapes and/or sizes in a regular fashion or randomly.

In various aspects, the electrodes on the surfaces of the jaw members may be made of metal. The exposed portions of the surfaces of the jaw members may have smooth surfaces to minimize sticking to tissue or coagulum and to facilitate their cleaning when tissue debris or coagulum does accumulate. The surfaces of the jaw members may include thermally conductive components such as copper, silver, aluminum, tungsten, nickel, or any other thermally conductive materials that may occur to those skilled in the art. Laminar composites coated with a biocompatible metal coating may be applied to the surfaces. The jaw members may include laminar composites of thermally conductive copper and a mechanically stronger material, particularly, higher modulus stainless steel. Biocompatibility of the jaw members may be maintained through an electro-deposited biocompatible metal coating, such as chromium, that coats both the stainless steel and copper laminate while not affecting the electrically insulative tissue engaging members. In some non-limiting examples, for end effectors with small jaw members, for example, having a width of about 0.039″ (1 mm) at their tip, laminar composites having a layer of 304 stainless steel of thickness of about 0.011″ and a corresponding layer of copper having about 0.052″ thickness may be provided. For larger jaw members, laminar composites having a layer of 304 stainless steel of thickness about 0.015″ and a corresponding layer of copper having about 0.075″ to about 0.085″ thickness may be provided. The biocompatible coating may be provided, for example, as an electro-deposited chromium coating, for example, that identified as MED-COAT 2000 marketed by Electrolyzing Corporation of Ohio, Cleveland, Ohio 44112. This biocompatible coating is described as meeting or exceeding USP Class VI certification.

The at least one electrically insulative tissue engaging member may be made of electrically insulative material. The electrically insulative material may be alumina, ceramic, nylon, polyphthalamide (PPA), TEFLON, polyimide, parylene, any other suitable material, and/or any combinations thereof. In various aspects, smooth metal surfaces may be provided on the surfaces of the jaw members to reduce sticking of tissue or coagulum and these surfaces may be coated with an electrically conductive non-stick coating. Upper surfaces of the at least one electrically insulative tissue engaging member may be coated with electrically insulative non-stick coating material. Such non-stick coating material may be sufficiently thin and/or applied to a sufficiently rough surface to provide a multiplicity of regions on the contacting surfaces that are uncoated with insulative non-stick coating material. Such non-stick coatings may include metal-filled (containing metal particles) organic materials such as fluoropolymers or other compounds generally known under the tradename TEFLON (polytetrafluoroethylene polymers and copolymers) (PTFE) or thin fluoropolymers known under the tradename VYDAX, both of which are manufactured by E.I. DuPont de Nemours of Wilmington, Del. In addition, metallic coatings such as ME-92 (ME-92 Operations, Providence, R.I.) and MED-COAT 2000 (supra) may be applied to the stainless steel surfaces of the jaw members to reduce the sticking of tissue thereto.

In various aspects, the length of the jaw members may be set for the particular application in surgery. For example, the length of the jaw members of about 0.4″ or 0.5″ to about 0.75″, such as about 0.47″ (12 mm), may be used for smaller anatomical structures or fine work. For larger anatomical structures, the length of the members may be about 1″ or greater, for example, about 1.57″ (40 mm).

The at least one electrically insulative tissue engaging member may have an appropriate diameter such that the electrically insulative tissue engaging member is neither so small as to pierce tissue nor so large as to take away too much of the electrode surface. The minimum diameter of the member may be about 0.03125″ ( 1/32″) as an electrically insulative tissue engaging member of this diameter may not pierce tissue unless the pressure applied on the tissue from the electrically insulative tissue engaging member is very high. If too much of the electrode surface is taken away by the electrically insulative tissue engaging member or members, there may be too little of the electrode surface and therefore, too little of the electrically conductive area adjacent to the electrically insulative tissue engaging member/members, and the electrosurgical device and/or the electrodes may not achieve the requisite performance. In some aspects where there is more than one electrically insulative tissue engaging member, the electrically insulative tissue engaging members may have the same or different diameters of any combination.

The at least one electrically insulative tissue engaging member may have a height about 0.001″ smaller than the gap between the electrodes or jaw members, for example, about 0.001″ to about 0.019″, preferably about 0.002″ to about 0.011″, more preferably about 0.003″ to about 0.009″, such as about 0.008″, about 0.003″ to about 0.007″, or about 0.004″ to about 0.007″. In general, the height may be less than about 0.020″ or less than or equal to about 0.010″. The minimum value found practical for the height may be about 0.003″. In some aspects where there is more than one electrically insulative member, the members may have the same or different heights of any combination.

These sizes may be selected to achieve the most efficient electrode contact geometry for achieving the most efficient hemostasis with respect to tissue or vessels grasped. The sizes and/or dimensions may be configured such that the electrosurgical device and the electrodes achieve the requisite performance.

In various aspects, the electrically insulative tissue engaging members may have the same height or different heights. The members may be provided on one jaw member and received in receiving pockets on the other jaw member. The depths of the receiving pockets may vary. The electrically insulative members and the receiving pockets may be configured to define a non-uniform arrangement along the length of the jaw members.

In various aspects, the electrically insulative tissue engaging members may be integrally made in the electrode. The electrically insulative tissue engaging members may be molded in the electrode(s). The electrically insulative tissue engaging members may be fabricated by an insert molding manufacturing process. This may reduce the cost of manufacturing. In some other aspects, the electrically insulative tissue engaging members may be inserted into openings defined by the electrode(s). In some other aspects, the electrode on the surface of one jaw member may be coined or bent to form tissue grasping members having the same function as the electrically insulative tissue engaging members that may contact a non-electrically conductive portion on the surface of the other jaw member. Portions on the surface of the other jaw member corresponding to the electrically insulative tissue engaging members may be cut out to expose the non-electrically conductive portion and receive the electrically insulative tissue engaging members. In some other aspects, the electrically insulative tissue engaging members may be made on a embossed insert that may be inserted in an insulated material in one jaw member. The embossed insert may be inserted and set in glue in the jaw member. A shim may be used to set the heights of the electrically insulative tissue engaging members.

In various aspects, the electrically insulative tissue engaging members may be made of ceramic, glass, or glass/ceramic applied by plasma deposition methods; physical vapor deposition; screen or pad printing followed by fusing of the insulative layer by exposure to high temperatures; a photolithography process; or attachment of individual ceramic members using brazing, soldering, or adhesive bonding methods. The electrically insulative tissue engaging members may be made from plastic and using coating methods such as, for example, dipping, plasma coating, encasement, or the like.

In some non-limiting examples, the electrically insulative tissue engaging members may be provided as discrete, spaced-apart elements disposed in arrays on one surface of a jaw member. The electrically insulative tissue engaging members may be cubes or any other suitable shapes. The insulative spacers defined within the arrays may be made by first depositing, for example, by plasma deposition or physical vapor deposition, an electrically insulative layer over a desired length of the surface. Next, thin grinding wheels can be used to grind away the electrically insulative layer to produce the pattern of electrically insulative tissue engaging members. In some non-limiting examples, the electrically insulative tissue engaging members or arrays may be made by thick film printing of insulative material followed by exposure to elevated temperatures to affect its bonding to the surface. In some non-limiting examples, the electrically insulative tissue engaging members may be made as layers utilizing a physical mask to deposit the electrically insulative material in required areas on the surface. Alternatively, the surface may be configured containing an array of openings of circular cross-sectional, peripheral shape, or any other suitable shape. The electrically insulative tissue engaging members may then be provided as electrically insulative glass, ceramic, or glass/ceramic pegs inserted within the openings.

In the following detailed description, reference is made to the accompanying drawings, which form a part hereof. In the drawings, similar symbols and reference characters typically identify similar components throughout the several views, unless context dictates otherwise.

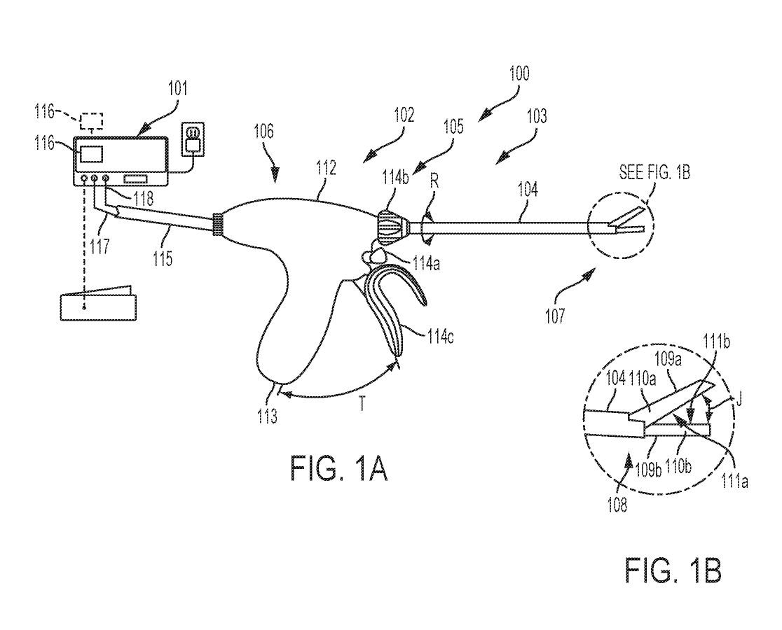

FIG. 1A shows an electrosurgical instrument 100 in electrical communication with a generator 101, according to one aspect of the present disclosure. The electrosurgical instrument 100 may be configurable with a flexible circuit 102 according to various aspects. The electrosurgical instrument 100 may comprise an elongate member 103, such as a shaft 104, having a proximal portion 105 coupled to a handle assembly 106. A distal portion 107 of the elongate member 103 may comprise an end effector 108 (see FIG. 1B) coupled to a distal end of the shaft 104. In some aspects, the end effector 108 may comprise a first jaw member 109a and a second jaw member 109b, each having an outer portion or surface 110a, 110b. At least one of the first jaw member 109a and the second jaw member 109b may move relative to the shaft 104. There may be only one jaw movable relative to the shaft 104, and the other jaw may be fixed relative to the shaft 104. At least one of the first jaw member 109a and the second jaw member 109b may be rotatably movable relative to the other along a path shown by arrow J to transition the first and second jaw members 109a, 109b between open and closed positions. In operation, the first and second jaw members 109a, 109b may be transitioned from the open position to a closed position to capture tissue therebetween. Captured tissue may contact one or more working portions of the jaw set 111a, 111b, configured to apply energy to treat target tissue located at or near the end effector 108. The type of energy may take various forms and includes, without limitation, monopolar and/or bipolar RF energy, microwave energy, reversible and/or irreversible electroporation energy, and/or ultrasonic energy, or any combination thereof. The handle assembly 106 may comprise a housing 112 defining a grip 113. In various aspects, the handle includes one or more control interfaces 114a-c, e.g., a button or switch 114a, rotation knob 114b rotatable along arrow R, and a trigger 114c movable relative to the grip 113 along arrow T, configured to provide operation instructions to the end effector 108. Multiple buttons, knobs, or triggers described also may be included as part of the housing 112 in order to manipulate one or more of the functioning members at the end effector 108. In some aspects, the handle assembly 106 may be further configured to electrically couple to a generator 101 to supply the electrosurgical instrument 100 with energy.

The generator 101 may be connected to the electrosurgical instrument 100 via a suitable transmission medium such as a cable 115. In one example, the generator 101 may be coupled to a controller, such as a control unit 116, for example. In various aspects, the control unit 116 may be made integrally with the generator 101, or may be provided as a separate circuit module or device electrically coupled to the generator 101 (shown in phantom to illustrate this option). The control unit 116 may include automated or manually operated controls to control the amount of current delivered by the generator 101 to the electrosurgical instrument 100. Although, as presently disclosed, the generator 101 is shown separate from the electrosurgical instrument 100, in some aspects, the generator 101 (and/or the control unit 116) may be made integrally with the electrosurgical instrument 100 to form a unitary electrosurgical system where a battery located within the electrosurgical instrument 100 may be the energy source and a circuit coupled to the battery produces the suitable electrical energy, ultrasonic energy, or heat energy. While the generator 101 is illustrated as generally coupled to the handle assembly 106, e.g., with a cord, it is to be understood that in some aspects the generator 101 may be positioned within the elongate member 103 and/or the handle assembly 106. For example, in one aspect, the generator 101 comprises one or more direct current batteries positioned in the handle assembly 106, shaft 104, or a portion thereof.

In one aspect, the generator 101 may comprise an input device located on a front panel of the generator 101. The input device may comprise any suitable device that generates signals suitable for programming the operation of the generator 101, such as a keyboard, or input port, for example. In one example, one or more electrodes in the first jaw member 109a and one or more electrodes in the second jaw member 109b may be coupled to the generator 101. The cable 115 may comprise multiple electrical conductors for the application of electrical energy to a first electrode (which may be designated as a + electrode) and to a second electrode (which may be designated as a − electrode) of the electrosurgical instrument 100. It may be recognized that + and − designations are made solely for convenience and do not indicate an electrical polarity. An end of each of the conductors may be placed in electrical communication with a terminal of the generator 101. The generator 101 may have multiple terminals, each configured to contact one or more of the conductors. The control unit 116 may be used to activate the generator 101, which may serve as an electrical source. In various aspects, the generator 101 may comprise an RF source, an ultrasonic source, a direct current source, and/or any other suitable type of electrical energy source, for example, one which may be activated independently or simultaneously. In various aspects, the cable 115 may comprise at least one supply conductor 117 and at least one return conductor 118, wherein current can be supplied to the electrosurgical instrument 100 via the at least one supply conductor 117 and wherein the current can flow back to the generator 101 via the at least one return conductor 118. In various aspects, the at least one supply conductor 117 and the at least one return conductor 118 may comprise insulated wires and/or any other suitable type of conductor. As described below, the at least one supply conductor 117 and the at least one return conductor 118 may be contained within and/or may comprise the cable 115 extending between, or at least partially between, the generator 101 and the end effector 108 of the electrosurgical instrument 100. The generator 101 can be configured to apply a sufficient voltage differential between the supply conductor 117 and the return conductor 118 such that sufficient current can be supplied to the end effector 108 to perform the intended electrosurgical operation.

In one example, the generator 101 may be implemented as an electrosurgery unit (ESU) capable of supplying power sufficient to perform bipolar electrosurgery using RF energy. In one example, the ESU can be a Force Triad™ Energy Platform sold by Medtronic of Boulder Colo. In some aspects, such as for bipolar electrosurgery applications, an electrosurgical instrument 100 having an active electrode and a return electrode can be utilized, wherein the active electrode and the return electrode can be positioned against, adjacent to, and/or in electrical communication with the tissue to be treated such that current can flow from the active electrode through the PTC bodies and to the return electrode through the tissue. Thus, in various aspects, the electrosurgical system may comprise a supply path and a return path, wherein the captured tissue being treated completes, or closes, the circuit. In other aspects, the generator 101 may provide sub-therapeutic RF energy levels for purposes of evaluating tissue conditions and providing feedback in the electrosurgical system. Such feedback may be employed to control the therapeutic RF energy output of the electrosurgical instrument 100. Sub-therapeutic RF energy levels may be used for bipolar surgical procedures if a risk analysis shows the possibility of neuromuscular stimulation has been mitigated to an acceptable level. Under some conditions, frequencies above 5 MHz may not be used in order to minimize problems associated with high frequency leakage currents. However, higher frequencies may be used in the case of bipolar techniques. It is generally recognized that 10 mA is the lower threshold of thermal effects on tissue.

During operation of electrosurgical instrument 100, the user generally grasps tissue, supplies energy to the grasped tissue to form a weld or a seal (e.g., by an actuating button and/or pedal), and then drives a tissue-cutting member at the distal end of the electrosurgical instrument through the grasped tissue. According to various aspects, a jaw-closing member may be provided, and the translation of the axial movement of the jaw-closing member may be paced, or otherwise controlled, to aid in driving the jaw-closing member at a suitable rate of travel. By controlling the rate of travel, the likelihood that the captured tissue has been properly and functionally sealed prior to transection with the cutting member may be increased.

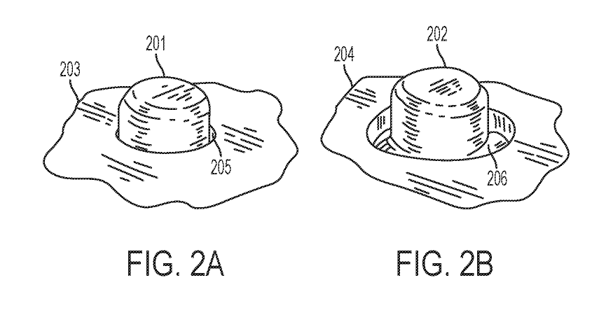

FIG. 2A and FIG. 2B show two types of electrically insulative tissue engaging members with respect to the electrode from which the members protrude, according to one aspect of the present disclosure. In FIG. 2A, an electrically insulative tissue engaging member 201 protrudes from an opening 205 defined by an electrode 203. The inner periphery of the opening 205 may be substantially the same as the outer periphery of the tissue engaging member 201. The opening 205 tightly fits around the tissue engaging member 201. The tissue engaging member 201 and the opening 205 may both have round peripheries as shown in the figure. However, it is understood that the tissue engaging member 201 and the opening 205 can have peripheries of any suitable shape or size. In FIG. 2B, an electrically insulative tissue engaging member 202 may protrude from an opening 206 defined by an electrode 204. The opening 206 may be larger than the tissue engaging member 202. The inner periphery of the opening 206 may not tightly fit the outer periphery of the tissue engaging member 202. The inner periphery of the opening 206 may be away from the outer periphery of the tissue engaging member 202 at a distance. The distance between the inner periphery of the opening 206 and the outer periphery of the tissue engaging member 202 may be uniform or non-uniform around the whole outer periphery of the tissue engaging member 202. The tissue engaging member 202 may or may not be at the center of the opening 206. The tissue engaging member 202 may have a round outer periphery. The opening 206 also may be round. The opening 206 may define a space in the form of a donut around the tissue engaging member 202.

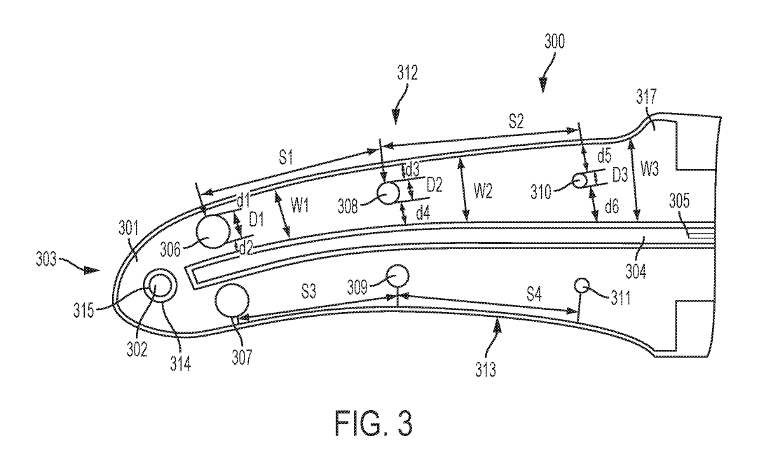

FIG. 3 shows a plan view of a jaw comprising a distal electrically conductive gap setting member 302 and electrically insulative tissue engaging members 306-311, according to one aspect of the present disclosure. An electrode 301 may be provided on one surface of the jaw member 300. An electrically conductive gap setting member 302 is located at the distal end 303 of the jaw member 300. In one aspect, the gap setting member 302 is a metal pin suitable to set a gap between the upper electrode (not shown) and the lower electrode 301. The gap setting member 302 is electrically conductive, but is electrically isolated from the surrounding electrode 301. In one aspect, the gap setting member 302 can be made of an electrically conductive metal or metal alloy and preferably can be made of steel, such as medical grade stainless steel, for example. The gap setting member 302 protrudes through an opening 314 defined by the lower electrode 301. A space 315 is provided between the inner periphery of the opening 314 and the outer periphery of the gap setting member 302 such that the gap setting member 302 does not contact any electrically conductive portion of the lower electrode 301. Although the opening 314 may have a substantially round shape, the opening 314 can have any shape as long as the inner periphery of the opening 314 may not contact any part of the outer periphery of the gap setting member 302. The gap setting member 302 may be located on the lower face of the upper jaw or on the upper face of the lower jaw member 300. The gap setting member 302 may be integrally made with or affixed on the lower electrode 301 or the upper electrode (not shown).

In one aspect, the electrically conductive gap setting member 302 can be made of an electrically conductive stiff material that resists deformation in response to an applied force. The material may be a high tensile strength incompressible material. The electrically conductive gap setting member 302 is located at the distal end 303 of the jaw member 300. In one aspect, the gap setting member 302 is an electrically conductive metal pin made of a stiff incompressible material having a high tensile strength, such as steel, and suitable for setting a gap between the upper electrode (not shown here) and the lower electrode 301. In one aspect, the gap setting member 302 can be made of an electrically conductive metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

A knife channel 304 may be provided in the interior, such as the middle, of the jaw member 300. A cutting member 305, such as a knife, may be provided in the channel 304 for cutting tissue after the tissue has been sealed using electrosurgical energy. The cutting member 305 may be slidable along the knife channel 304. The cutting member 305 may be adapted to cut tissue by moving distally in the knife channel 304 when the jaw member 300 and the opposing jaw are in the closed position to grip tissue. Although generally speaking the knife channel 304 may be located along the lateral center of the electrode 301, this is not necessarily always the case. Thus, in other aspects, the knife channel 304 may be offset from the center to either side of the electrode 301.

At least one electrically insulative tissue engaging member may be located on the lower electrode 301 of the jaw member 300, for example, to provide grasping surfaces to grasp tissue. The at least one electrically insulative tissue engaging member 306-311 provided on at least one of two sides 312, 313 of the electrode 301 along the knife channel 304. The tissue engaging members may change shape along the length of the electrode 301. The tissue engaging members 306-311 may change shape down the length of the electrode 301 from the distal end 303 to the proximal end 317 of the jaw member 300. For example, the tissue engaging members 306-311 may be cylinders, and therefore, the upper surfaces of the tissue engaging members 306-311 may be round. The top of the tissue engaging members 306-311 also may provide grasping surfaces to grasp tissue.

The tissue engaging members 306-311 may be located on an insulative element of the jaw member 300 that supports the lower electrode 301. In this configuration, the tissue engaging members 306-311 protrude through the lower electrode 301. In another aspect, the tissue engaging members 306-311 may be located on an insulative element of the upper jaw member that supports the upper electrode 503. In this configuration, the tissue engaging members 306-311 protrude through the upper electrode. Accordingly, either or both upper and/or lower electrodes may comprise tissue engaging members 306-311 configured to provide grasping surfaces to grasp tissue. The top of the tissue engaging members 306-311 also may be configured to provide grasping surfaces to grasp tissue.

The electrically insulative tissue engaging members 306-311 may be made of electrically insulative material such as a polymer and, more specifically, can be an electrically insulative material (e.g., polyimide, polyester, fluorocarbon, or any polymeric material, or any combinations thereof). The tissue engaging members 306-311 may be generally attached to the tissue contacting side of the jaw member 300.

In one aspect, the tissue engaging members 306-311 may comprise a nonstick coating or may be made of a nonstick material such as TEFLON. Any nonstick material or nonstick surface finish may be suitable to prevent tissue from sticking to the electrically conductive portion of the electrode 301. The electrically insulative material of the tissue engaging members 306-311 may be alumina, ceramic, nylon, polyphthalamide (PPA), TEFLON, polyimide, parylene, any other suitable material, and/or any combinations thereof. Top surfaces of the tissue engaging members 306-311 may be coated with electrically insulative non-stick coating material. Such non-stick coating material may be sufficiently thin and/or applied to a sufficiently rough surface to provide a multiplicity of regions on the contacting surfaces that are uncoated with insulative non-stick coating material. Such non-stick coatings may include metal-filled (containing metal particles) organic materials such as fluoropolymers or other compounds generally known under the tradename TEFLON (polytetrafluoroethylene polymers and copolymers) or thin fluoropolymers generally known under the tradename VYDAX, both of which are manufactured by E.I. DuPont de Nemours of Wilmington, Del. In addition, metallic coatings such as ME-92 (ME-92 Operations, Providence, R.I.) and MED-COAT 2000 (supra) may be applied to the stainless steel surfaces of the jaw member 300 to reduce the sticking of tissue thereto. In one aspect, the electrically insulative tissue engaging member 306-311 may be made of a dielectric material that can be printed on the electrodes. In one aspect, the tissue engaging members 306-311 comprises a nonstick coating or may be made of a nonstick material such as polytetrafluoroethylene (PTFE), which is a synthetic fluoropolymer of tetrafluoroethylene that has numerous applications. The best known trade name of PTFE-based formulas may be TEFLON by DuPont Co., for example.

In one aspect, an electrically insulative tissue engaging layer may be made by bonding a dielectric cover film on the tissue contacting surface of the electrode 301. The electrically insulative tissue engaging members 306-311 may be made by etching the dielectric cover film bonded to the tissue contacting surface of the electrode 301. In one aspect, the tissue engaging members 306-311 may be made of electrically insulative material such as a polymer, more specifically a polyimide, polyester, fluorocarbon, or any polymeric material, or any combinations thereof. The tissue engaging members 306-311 may be generally attached to the tissue contacting surface of the upper jaw member or lower jaw member 300.

At each side 312, 313 of the lower electrode 301, the widths of the side of the electrode 301 increase down the length of the electrode from the distal end 303 to the proximal end 317 of the jaw member 300. For example, the widths W1-W3 of the side 312 of the electrode 301 increase down the length of the electrode 301 from the distal end 303 to the proximal end 317 of the jaw member 300. Distances from the members to the edges of the sides of the electrode also may change down the length of the electrode 301 from the distal end 303 to the proximal end 317 of the jaw member 300. For example, the distances d1-d6 from the members 306, 308, 310 to the edges of the side 312 of the electrode 301 may increase down the length of the electrode 301 from the distal end 303 to the proximal end 317 of the jaw member 300. For example, d1, d2<d3, d4<d5, d6. The distances d1 and d2 may be the same or different. The distances d3 and d4 may be the same or different. The distances d5 and d6 may be the same or different. The spacing S1-S4 between the members may be the same or different. The spacing S1-S4 may be selected to achieve the most efficient contact geometry for, in turn, achieving the most efficient hemostasis with respect to tissue or vessels grasped between the jaws.

Along each side 312, 313 of the lower electrode 301, sizes of the upper surfaces of the members may increase, decrease, or change randomly down the length of the electrode from the distal end 303 to the proximal end 317 of the jaw member 300. For example, as shown here, diameters D1-D3 of the members 306, 308, and 310 along the side 312 of the electrode may decrease down the length of the lower electrode 301 from the distal end 303 to the proximal end 317 of the jaw member 300.

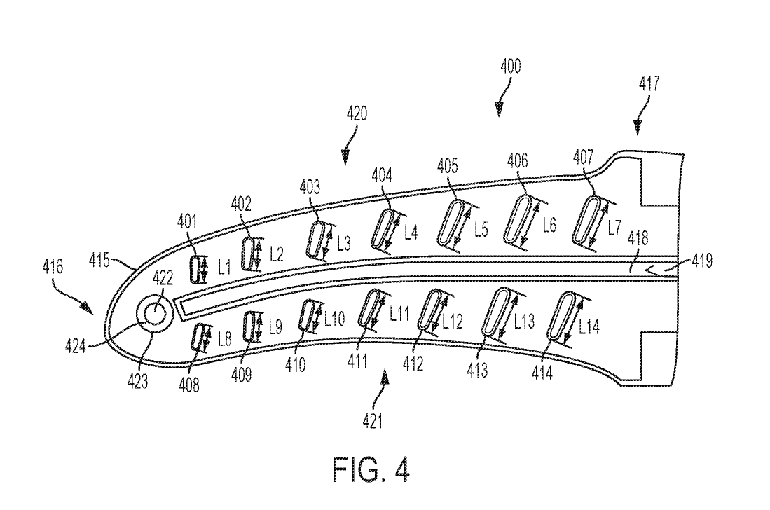

FIG. 4 shows a plan view of a jaw member 400 comprising a distal electrically conductive gap setting member 422 and electrically insulative tissue engaging members 401-414, according to one aspect of the present disclosure. The jaw member 400 may include electrically insulative tissue engaging members 401-414 along the lower electrode 415 between the distal end 416 and the proximal end 417 of the jaw member 400. The jaw member 400 may include a channel 418 in the middle of the jaw. A cutting member 419, such as a knife, may be provided in the channel 418 for cutting tissue. The cutting member 419 may be slidable along the channel 418.

The electrically insulative tissue engaging members 401-407 may be provided on one side 420 of the lower electrode 415. The tissue engaging members 408-414 may be provided on the other side 421 of the lower electrode 415. The tissue engaging members 401-414 may be in the form of elongate members. The tissue engaging members 401-414 may be oriented in any direction. The tissue engaging members 401-414 may be provided parallel to each other. The tissue engaging members 401-414 may extend between the peripheries of the lower electrode 415. The tissue engaging members 401-414 may have a uniform or different thickness T. The tissue engaging members 401-414 may be evenly or non-evenly spaced along the length of the lower electrode 415. The distance S between the tissue engaging members 401-414 may be the same or different. The sizes and/or the shapes of the tissue engaging members 401-414 may change along the lower electrode 415 between the distal end 416 and the proximal end 417 of the jaw member 400. For example, the lengths L1-L7 along the side 420 of the electrode 415 may increase down the length of the electrode 415 from the distal end 416 to the proximal end 417 of the jaw member 400. The lengths L8-L14 along the side 412 of the lower electrode 415 also may increase down the length of the electrode 415 from the distal end 416 to the proximal end 417 of the jaw member 400. The lengths of the tissue engaging members 401-414 along one side of the electrode 415 also may decrease or change randomly down the length of the electrode 415 from the distal end 416 to the proximal end 417 of the jaw member 400, which is not shown here.

The electrically insulative tissue engaging members 401-414 may be located on an insulative element of the lower jaw member 400 that supports the lower electrode 415. In this configuration, the tissue engaging members 401-414 protrude through the lower electrode 415. In another aspect, the tissue engaging members 401-414 may be located on an insulative element of an upper jaw member that supports an upper electrode (not shown here). In this configuration, the tissue engaging members 401-414 protrude through the upper electrode. Accordingly, either or both upper electrode and/or lower electrodes 415 may comprise tissue engaging members 401-414 configured to provide grasping surfaces to grasp tissue located between the upper electrode and lower electrode 415. The top of the tissue engaging members 401-414 also may be configured to provide grasping surfaces to grasp tissue.

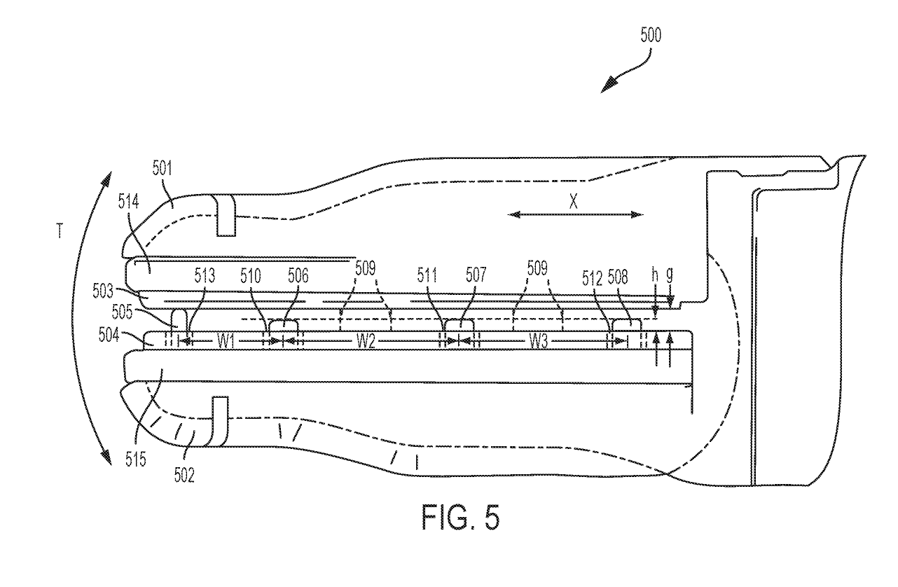

In one aspect, the electrically insulative tissue engaging members 401-414 are made of an electrically insulative material as described generally hereinabove and particularly in connection with FIGS. 3 and 5. For example, the tissue engaging members 401-414 may be made of electrically insulative material such as a polymer and, more specifically, can be an electrically insulative material (e.g., polyimide, polyester, fluorocarbon, or any polymeric material, or any combinations thereof). The tissue engaging members 401-414 may be generally attached to the tissue contacting side of the jaw member 400. In one aspect, the tissue engaging members 401-414 may comprise a nonstick coating or may be made of a nonstick material such as TEFLON. Any nonstick material or nonstick surface finish may be suitable to prevent tissue from sticking to the electrically conductive portion of the upper electrode (not shown).

An electrically conductive gap setting member 422 is located at the distal end 416 of the jaw member 400. In one aspect, the gap setting member 422 is an electrically conductive metal pin made of a stiff incompressible material having a high tensile strength, such as steel, and suitable for setting a gap between the upper electrode (not shown) and the lower electrode 415. In one aspect, the gap setting member 422 can be made of an electrically conductive metal or metal alloy and preferably can be made of steel, such as medical grade stainless steel, for example. The gap setting member 422 may protrude through an opening 423 defined by the electrode 415. A space 424 is provided between the inner periphery of the opening 423 and the outer periphery of the gap setting member 422 such that the gap setting member 422 does not contact any electrically conductive portion of the electrode 415. Although the opening 423 is shown to have a substantially round shape, the opening 423 can have any shape as long as the inner periphery of the opening 423 may not contact any part of the outer periphery of the gap setting member 422.

The electrically conductive gap setting member 422 is located at the distal end 416 of the jaw member 400. The gap setting member 422 may protrude through an opening 423 defined in the electrode 415. A space 424 is provided between the inner periphery of the opening 423 and the outer periphery of the gap setting member 422 such that the gap setting member 422 does not contact any electrically conductive portion of the electrode 415. Although the opening 423 may have a substantially round shape, the opening 423 can have any shape as long as the inner periphery of the opening 423 may not contact any part of the outer periphery of the gap setting member 422.

In one aspect, the electrically conductive gap setting member 422 is an electrically conductive metal pin made of a stiff incompressible material having a high tensile strength and suitable for setting a gap between an upper electrode (not shown here) and a lower electrode 415. In one aspect, the gap setting member 422 is made of an electrically conductive stiff incompressible material having a high tensile strength as described generally hereinabove and particularly in connection with FIG. 3 and hereinbelow in connection with FIG. 5. For example, the gap setting member 422 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

FIG. 5 shows a side elevational view of one aspect of an end effector 500 comprising a distal electrically conductive gap setting member 505 and electrically insulative tissue engaging members 506, 507, 508 located, according to one aspect of the present disclosure. The end effector 500 may have an upper jaw member 501 and a lower jaw member 502. At least one of the upper jaw member 501 and the lower jaw member 502 may move relative to the other. Both the upper jaw member 501 and the lower jaw member 502 may move relative to each other. At least one of the upper jaw member 501 and the lower jaw member 502 may move along the arrow T. The upper jaw member 501 and the lower jaw member 502 may have an open position and a closed position. In the open position, the upper jaw member 501 and the lower jaw member 502 may have the maximum distance from each other and may not move farther away from each other. In the closed position, the upper jaw member 501 and the lower jaw member 502 may have the minimum distance from each other and may not move closer to each other. The movement of at least one of the jaw members 501, 502 may bring the upper and lower jaw members 501, 502 to a position between the open position and the closed position.

At least one electrode is positioned on at least one jaw member. An upper electrode 503 is provided on a lower surface of the upper jaw member 501. A lower electrode 504 is positioned on an upper surface of the lower jaw member 502. The upper and lower electrodes 503, 504 may be provided facing each other. An electrically conductive gap setting member 505 is located at the distal end of the end effector 500. The gap setting member 505 is electrically conductive and in one aspect is a pin made of a stiff incompressible material having a high tensile strength. In one aspect, gap setting member 505 can be made of a metal or metal alloy and preferably can be made of steel, such as medical grade stainless steel, for example, suitable for setting a gap between the upper and lower electrodes 503, 504. The gap setting member 505 may be provided on the lower face of the upper jaw member 501 or on the upper face of the lower jaw member 502. The gap setting member 505 may be made with or affixed on the lower jaw member 502 or the upper jaw member 501. The gap setting member 505 protrudes through an opening 511 defined by the lower electrode 503 without contacting the lower electrode 503. A space is provided between the inner periphery of the opening 511 and the outer periphery of the gap setting member 505 such that the gap setting member 505 does not contact any electrically conductive portion of the lower electrode 504. Although the opening 511 may have a substantially round shape, the opening 511 can have any shape as long as the inner periphery of the opening 511 may not contact any part of the outer periphery of the gap setting member 505.

A gap “g” is defined as a minimum distance or space between the electrodes 503, 504 when the upper and lower jaw members 501, 502 are in the closed position. As shown here, the gap “g” may be substantially uniform along the length of the electrodes 503, 504 and/or along the length of the jaw members 501, 502. The gap “g” also may be non-uniform, which is not shown. The gap “g” is defined by the gap setting member 505. The gap setting member 505 prevents the undesired contact between the upper and lower electrode 504, 504 when the jaw members 501, 502 are in the closed position.

In one aspect, the gap setting member 505 is an electrically conductive metal pin made of a stiff incompressible material having a high tensile strength, such as steel, and suitable for setting a gap between the upper electrode 503 and the lower electrode 504. In one aspect, the gap setting member 505 can be made of an electrically conductive metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

At least one of the upper or lower jaw members 501, 502 comprises an electrically insulative tissue engaging layer or element to facilitate grasping or gripping tissue located between the upper and lower jaw members 501, 502. Electrically insulative tissue engaging members 506-508 may be provided on at least one of the upper or lower jaw members 501, 502. In one aspect, the tissue engaging members 506-508 protrude through corresponding openings 512-514 defined by the lower electrode 504 of the lower jaw member 502. Although the tissue engaging members 506-508 are provided on the upper surface of the lower jaw member 502, in other aspects, electrically insulative tissue engaging members may be provided on a lower surface of the upper jaw member 501 and preferably protrude through openings above the surfaces of the upper and lower electrodes 503, 504 to provide grasping surfaces to grasp tissue located between the jaw members 501, 502. The top of the tissue engaging members 506-508 provide grasping surfaces to grasp tissue.

The electrically insulative tissue engaging members 506-508 may be located on an insulative element 515 of the lower jaw member 502 that supports the lower electrode 504. In this configuration, the tissue engaging members 506-508 protrude through corresponding openings 512-514 defined by the lower electrode 504 of the lower jaw member 502. In another aspect, the tissue engaging members 506-508 may be located on an insulative element 514 of the upper jaw member 501 that supports the upper electrode 503. In this configuration, the tissue engaging members 506-508 protrude through corresponding openings (not shown) defined by the upper electrode 503. Accordingly, either or both upper and/or lower electrodes 503, 504 may comprise tissue engaging members 506-508 configured to provide grasping surfaces to grasp tissue. The top of the tissue engaging members 506-508 also may be configured to provide grasping surfaces to grasp tissue.

The at least one electrically insulative tissue engaging member 506-508 may be made of electrically insulative material. The electrically insulative material may be alumina, ceramic, nylon, polyphthalamide (PPA), TEFLON, polyimide, parylene, any other suitable material, and/or any combinations thereof. In various aspects, smooth metal surfaces may be provided on the surfaces of the jaw members to reduce sticking of tissue or coagulum and these surfaces may be coated with an electrically conductive non-stick coating. Upper surfaces of the at least one electrically insulative tissue engaging member may be coated with electrically insulative non-stick coating material. Such non-stick coating material may be sufficiently thin and/or applied to a sufficiently rough surface to provide a multiplicity of regions on the contacting surfaces that are uncoated with insulative non-stick coating material. Such non-stick coatings may include metal-filled (containing metal particles) organic materials such as fluoropolymers or other compounds generally known under the tradename TEFLON (polytetrafluoroethylene polymers and copolymers) (PTFE) or thin fluoropolymers known under the tradename VYDAX, both of which are manufactured by E.I. DuPont de Nemours of Wilmington, Del. In addition, metallic coatings such as ME-92 (ME-92 Operations, Providence, R.I.) and MED-COAT 2000 (supra) may be applied to the stainless steel surfaces of the jaw members to reduce the sticking of tissue thereto.

In one aspect, the electrically insulative tissue engaging members 506-508 may be made of a dielectric material that can be printed on either the upper or lower electrodes 503, 504. In yet another aspect, the electrically insulative layer may be configured as an electrically insulative cover that further defines the electrically conductive jaw electrode 503, 504 and can act as a tissue engaging member. In one aspect, the tissue engaging members 506-508 may comprise a nonstick coating or may be made of a nonstick material such as polytetrafluoroethylene (PTFE), which is a synthetic fluoropolymer of tetrafluoroethylene that has numerous applications. PTFE-based formulas are best known under the may be tradename TEFLON by DuPont Co., for example. In one aspect, the tissue engaging members 506, 507, and 508 may be made of a dielectric material.

In one aspect, an electrically insulative tissue engaging layer may be made by bonding a dielectric cover film on the tissue contacting surface of the upper and lower electrodes 503, 504. In one aspect, the electrically insulative tissue engaging members 506-508 may be made by etching the dielectric cover film bonded to the tissue contacting surface of the upper or lower electrode 503, 504. In one aspect, at least one of the tissue engaging members 506-508 may be configured to facilitate gripping, grasping, or otherwise manipulating tissue located between the upper and lower electrodes 503, 504.

In one aspect, the electrically insulative tissue engaging members 506-508 may be made of electrically insulative material such as a polymer, more specifically a polyimide, polyester, fluorocarbon, or any polymeric material, or any combinations thereof. The tissue engaging members 506-508 may be attached to the tissue contacting surface of the upper or lower jaw members 501, 502.

In one aspect, the upper and lower electrodes 503, 504 can be mass produced for a bipolar medical device, generally referred to as an electrosurgical device. A flexible electrically conductive sheet (e.g., Cu) may be bonded to an electrically insulative backing sheet (e.g., polyimide backing), and the electrically insulative tissue engaging members 506-508 may be printed at two or more locations on at least one of the upper and lower electrodes 503, 504. The tissue engaging members 506-508 may serve to assist or facilitate gripping, grasping, or manipulating tissue located between the upper and lower electrodes 503, 504 of the upper and lower jaw members 501, 502.

In one aspect, the upper and lower electrodes 503, 504 can be produced by laminating the metallic sheet to an electrically insulative film made of polyimide, polyester, fluorocarbon, or any polymeric material, or any combinations thereof. The electrically insulative layer, as well as the electrically insulative tissue engaging members 506-508, may be screen printed on the electrically conductive face of the upper and lower electrodes 503, 504. The shape of the upper and lower electrodes 503, 504 may be made by screen printing a protective barrier to the metallic film. This protective barrier may allow the shape of the upper and lower electrodes 503, 504 to be made by photoetching away the remaining material that does not make up the final shape of the upper and lower electrodes 503, 504. Finally, the individual electrode may be die-cut leaving an electrode subassembly that can be bonded to the upper and lower jaw members 501, 502.

The electrically insulative tissue engaging members 506-508 can have an adhesive or a brazable surface on the back side to attach the upper or lower electrode 503, 504 to the corresponding upper or lower jaw member 501, 502 of the end effector 500 depending on the jaw construction of the electrosurgical instrument 100 (FIG. 1).

Further, the upper and lower electrodes 503, 504 may be made of the following materials having the indicated thicknesses: copper, gold plated copper, silver, platinum, stainless steel, aluminum, or any suitable electrically conductive biocompatible material, among other electrically conductive metals and/or alloys. In one example, the upper and lower electrodes 503, 504 can include an electrically conductive metal layer (e.g., copper, gold plated copper, silver, platinum, stainless steel, aluminum, or any suitable electrically conductive biocompatible material, for example, among other electrically conductive metals and/or alloys), an electrically insulative film (e.g., polyimide, polyester, fluorocarbon, or any polymeric material, or any combinations thereof) bonded to the electrically conductive metal layer, and an adhesive used to bond the electrically conductive metal layer to the electrically insulative film.

In one example, the upper and lower electrode 503, 504 may comprise an acrylic-based copper clad laminate known under the tradename PYRALUX LF9250 supplied by DuPont, the copper clad laminate comprising a coverlay, a bondply, and a sheet adhesive. A coverlay may be a material laminated to the outside layers of the circuit to insulate the copper conductor. A bondply may be an adhesive system of unreinforced, thermoset based thin film available in various thicknesses intended for use in high performance, high reliability multi-layer flexible circuit constructions. In one aspect, the components of the upper and lower electrode 503, 504 may comprise a copper layer having a thickness of about 0.0028″, a polyimide film layer having a thickness of about 0.005″, and an adhesive layer having a thickness of about 0.001″, for a total thickness of about 0.0088″. In one aspect, the upper and lower electrode 503, 504 may comprise a copper layer having a thickness of about 0.0028″, a polyimide film layer having a thickness of about 0.003″, and an adhesive layer having a thickness of about 0.001″ for a total thickness of about 0.0068″. It will be appreciated that the thicknesses of the individual layers and the total thickness may vary based on the particular implementation details.

The various types of electrodes and electrically insulative tissue engaging members described in connection with the other figures herein can be manufactured in a manner similar to that described in the preceding paragraphs and for conciseness and clarity of disclosure will not be repeated in the description of such figures.

As used throughout this description, the electrically conductive gap setting member 505 may be a piece of material used to create or maintain a space between two things, such as upper and lower jaw members 501, 502 of the end effector 500. The gap setting member 505 can be made of a material that is electrically conductive and also is stiff to resist deformation in response to an applied force. The material is stiff with a high tensile strength and is incompressible. The gap setting member 505 is electrically conductive. In one aspect, the electrically conductive gap setting member can be made of an electrically conductive metal or metal alloy and preferably can be made of steel, such as medical grade stainless steel, for example. Alternatively, the gap setting member 505 can be made of exotic materials, including platinum, molybdenum disilicide, and silicon carbide provided that these materials have a suitable electrical conductivity. These are just a few examples, which are not meant to be limiting. In an electrically conductive configuration, the gap setting member 505 may be employed to set a uniform or non-uniform predetermined gap between tissue contacting surfaces of the electrodes 503, 504 of the upper and lower jaw members 501, 502.

The electrically insulative tissue engaging members 506-508 each may have the same height or different heights. The tissue engaging members 506-508 are provided for facilitating or assisting gripping, grasping, or otherwise manipulating tissue and thus do not contact the opposing electrode. In one aspect, the tissue engaging members 506-508 have a dimension or height that is less than the dimension or height of the electrically conductive gap setting member 505. The tissue engaging members 506-508 may have a height or heights different from the height of the gap setting member 505. The tissue engaging members 506-508 each may have the same height “h”. The value of height “h” is smaller than the distance “g” set by the gap setting member 505, which may be the same as the size of the gap or the distance between the jaw members 501, 502 when the gap is uniform. Along the longitudinal axis “x”, the distances W1, W2, W3 between the centers of the gap setting member 505 and the tissue engaging members 506-508 may be the same or different. The heights of the tissue engaging members 506-508 are selected such that the tissue or vessel media may be securely grasped and extruded into the recesses between the tissue engaging members 506-508 to assure electrical contact with the grasping surfaces. This develops a current flux flow path as represented, for example, at dashed lines 509, which extend directly across the surfaces of the upper and lower electrodes 503, 504, enhancing the most efficient hemostasis geometry for the instrument. Such extrusion of the tissue or vessel media into the recesses between the members may serve to achieve a secure grasping and sealing thereof during its surgical manipulation and throughout the coagulation process. The height of the gap setting member 505 may be selected to set a gap “g” that is greater than the height “h” and to facilitate or assist the application of a suitable pressure to the tissue when the upper and lower jaw members 501, 502 are in a closed position to promote an adequate tissue seal.

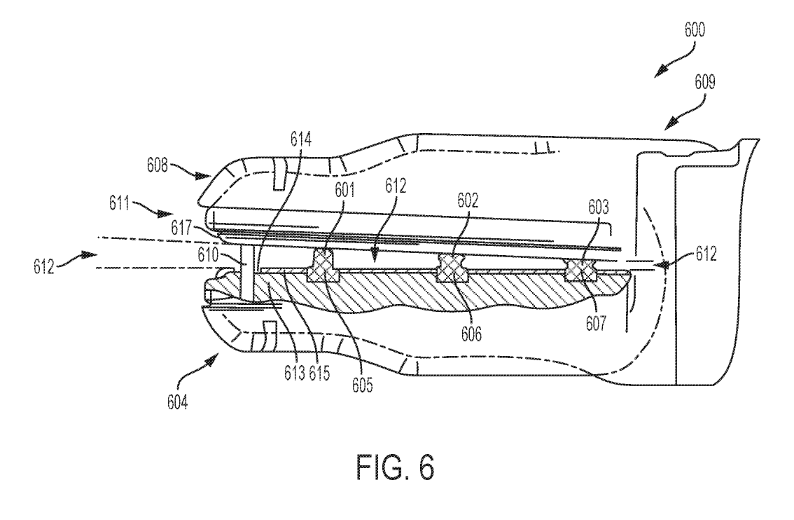

FIG. 6 shows a side elevational view of one aspect of an end effector 600 comprising a distal electrically conductive gap setting member 610 and electrically insulative tissue engaging members 601, 602, 603 having non-uniform stiffness, according to one aspect of the present disclosure. The tissue engaging members 601-603 are protuberances in the lower jaw member 604 of the end effector 600. The protuberances protrude through corresponding openings 605, 606, 607 defined by the lower electrode 615. The tissue engaging members 601-603 may be linearly or non-linearly progressing along the lower jaw member 604. The tissue engaging members 601-603 have a non-uniform stiffness. For example, the tissue engaging member 601 may be stiff. The tissue engaging member 602 may have medium stiffness. The tissue engaging member 603 may be compliant. When the upper jaw member 608 presses down on the tissue engaging members 601-603, the members 601-603 may deform to different extents. The tissue engaging member 601 may not deform or may have the least deformation among the members 601-603. The tissue engaging member 602 may deform slightly. The tissue engaging member 603 may deform the most. The terms such as “least,” “slightly,” and “most” refer to relatively different extents when the tissue engaging members 601-603 are compared. It is understood that there may be less or more than three members provided in the end effector according to the present disclosure. The three electrically insulative tissue engaging members 601-603 described herein are merely one aspect for illustration purposes. Generally, the closer an electrically insulative tissue engaging member may be to the proximal end 609 of the end effector 600, the more compliant the tissue engaging member may be configured and the more deformed the electrically insulative tissue engaging member may be when pressed by the opposing jaw member.

The electrically insulative tissue engaging member 601 may be made of a stiff material that as a stiffness less than the stiffness of the electrically conductive gap setting member 610. The tissue engaging member 602 may be made of a material of medium stiffness. The electrically insulative tissue engaging member 603 may be made of a compliant material, including but not limited to nylon 10, nylon 12, nylon 66, or any other suitable material. Some other suitable materials may include silicon rubber and/or urethane configured to have the desired stiffness for the electrically insulative tissue engaging members. Materials suitable herein may be selected based on their temperature withstanding capabilities, dielectric properties, bio-compatibilities, and any other factor that may be recognized by one skilled in the art. For example, the suitable material may have desired arc-tracking resistance. One material that has good arc-tracking resistance may be Grivory. Some other suitable materials may include Thermec (nylon), Amodel (PPA), and Dupont Zytel (nylon).

Alternatively, the electrically insulative tissue engaging members 601-603 are made of an electrically insulative material as described generally hereinabove and particularly in connection with FIGS. 3 and 5. For example, the tissue engaging members 601-603 may be made of electrically insulative material such as a polymer and, more specifically, can be an electrically insulative material (e.g., polyimide, polyester, fluorocarbon, or any polymeric material, or any combinations thereof). The tissue engaging members 601-603 may be generally attached to the tissue contacting side of the lower jaw member 604. In one aspect, the tissue engaging members 601-603 may comprise a nonstick coating or may be made of a nonstick material such as TEFLON. Any nonstick material or nonstick surface finish may be suitable to prevent tissue from sticking to the electrically conductive portion of the upper electrode 617.

An electrically conductive gap setting member 610 is located at the distal end 611 of the end effector 600. The gap setting member 610 may protrude through an opening 613 defined by the lower electrode 615. There may be a space 614 between the inner periphery of the opening 613 and the outer periphery of the gap setting member 610 such that the gap setting member 610 may not contact any electrically conductive portion of the lower electrode 615. Although the opening 613 is shown to have a substantially round shape, the opening 613 can have any shape as long as the inner periphery of the opening 613 may not contact the outer periphery of the gap setting member 610. The gap setting member 610 may define a non-uniform gap 612 between the jaw members 604, 608 by itself or together with the electrically insulative members 601-603 or other elements or techniques. The gap setting member 610 may be configured to form the non-uniform gap 612 between the upper and lower electrodes 617, 615 when the upper and lower jaw members 604, 608 in the closed position. Generally, a non-uniform gap may be defined by a minimum distance and a maximum distance between the upper and lower electrodes 617, 615 where the distance between the upper and lower electrodes 617, 615 varies linearly or non-linearly between the minimum and the maximum distance.

In one aspect, the electrically conductive gap setting member 610 is an electrically conductive metal pin made of a stiff incompressible material having a high tensile strength and suitable for setting a non-uniform gap 612 between the upper and lower electrodes 617, 615. In one aspect, the gap setting member 610 is made of an electrically conductive stiff incompressible material having a high tensile strength as described generally hereinabove and particularly in connection with FIGS. 3 and 5. For example, the gap setting member 610 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

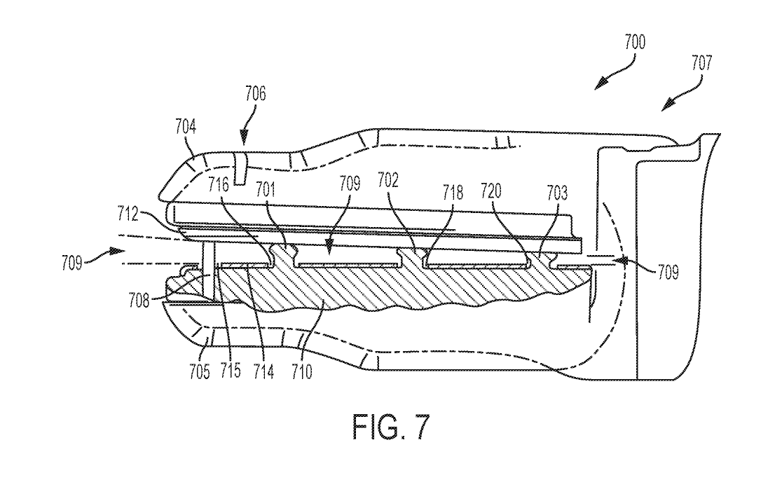

In another aspect, FIG. 7 shows an end effector 700 comprising a distal electrically conductive gap setting member 708 and compliant electrically insulative tissue engaging members 701, 702, 703, according to one aspect of the present disclosure. As shown in FIG. 7, the tissue engaging members 701-703 all may be compliant. The tissue engaging members 701-703 protrude through corresponding openings 716, 718, 720 defined by the lower electrode 714. An electrically conductive gap setting member 708 is located at the distal end of the end effector 700. An opening 715 is defined about the gap setting member 708 to electrically isolate the gap setting member 708 from the lower electrode 714. When a force 706 is applied to at least one of the upper or lower jaw members 704, 705 to close the upper and lower jaw members 704, 705 toward the closed position, the tissue engaging members 701-703 may deform under the pressure from the opposing jaw member 704. The closer to the proximal end 707 of the end effector 700, the more deformed the tissue engaging members 701-703 may be. When deformed, the tissue engaging members 701-703 may form mushroom heads at varying heights. When the tissue engaging upper and lower jaw members 704, 705 are in the closed position, along with the gap setting member 708, the tissue engaging members 701-703 may be deformed at varying heights to form a non-uniform gap 709. The tissue engaging members 701-703 may be molded together with the inner portion 710 of the lower jaw member 705. The tissue engaging members 701-703 and the inner portion 710 of the lower jaw member 705 may be composed of the same material and may be made in one piece of such material.

In one aspect, the electrically insulative tissue engaging members 701-703 are made of an electrically insulative material as described generally hereinabove and particularly in connection with FIGS. 3 and 5. For example, the tissue engaging members 701-703 may be made of electrically insulative material such as a polymer and, more specifically, can be an electrically insulative material (e.g., polyimide, polyester, fluorocarbon, or any polymeric material, or any combinations thereof). The tissue engaging members 701-703 may be generally attached to the tissue contacting side of the upper jaw member 704. In one aspect, the tissue engaging members 701-703 may comprise a nonstick coating or may be made of a nonstick material such as TEFLON. Any nonstick material or nonstick surface finish may be suitable to prevent tissue from sticking to the electrically conductive portion of the upper electrode 712.

In one aspect, the electrically conductive gap setting member 608 is an electrically conductive metal pin made of a stiff incompressible material having a high tensile strength and suitable for setting a non-uniform gap 709 between the upper and lower electrodes 712, 714. In one aspect, the gap setting member 708 is made of an electrically conductive stiff incompressible material having a high tensile strength as described generally hereinabove and particularly in connection with FIGS. 3 and 5. For example, the gap setting member 708 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

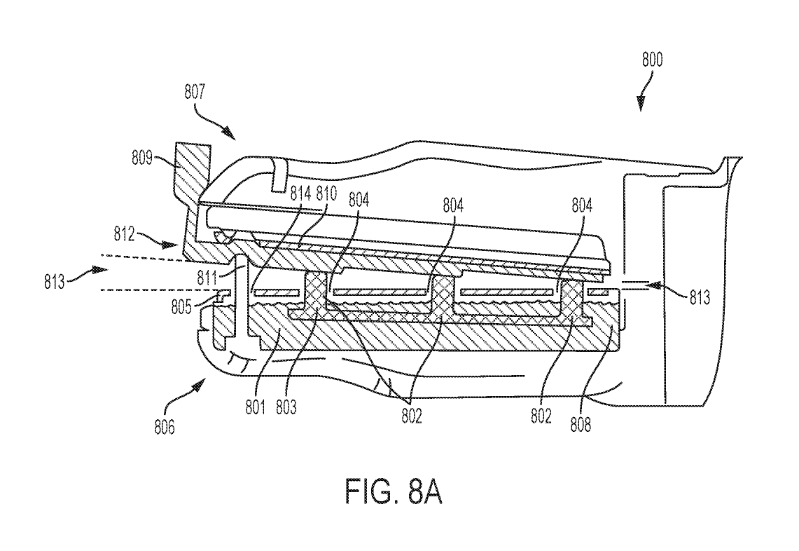

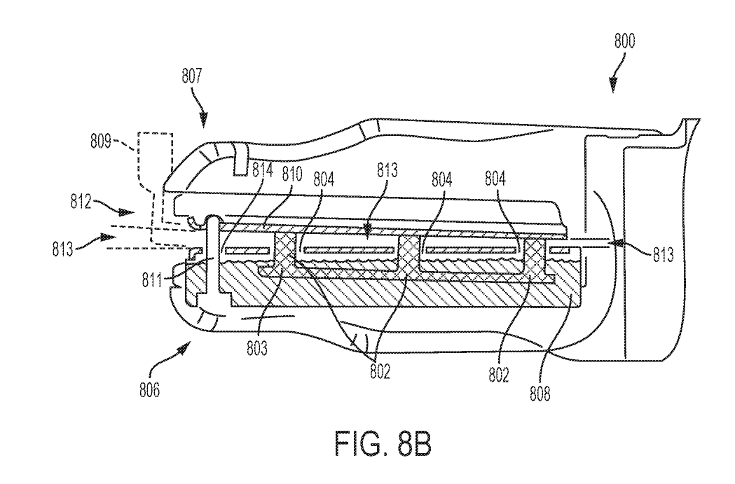

FIG. 8A and FIG. 8B show one example process of manufacturing an end effector 800 comprising a distal electrically conductive gap setting members 811 and an electrically insulative tissue engaging member assembly 802, according to one aspect of the present disclosure. The electrically insulative tissue engaging member assembly 801 may include electrically insulative tissue engaging member assembly 802 provided on an embossed insert 803. The tissue engaging member assembly 802 may extend from the lower of the embossed insert 803 and protrude through openings 804 defined by the lower electrode 805 on the lower jaw member 806. The embossed insert 803 may be inserted in the inner portion 808 of the lower jaw member 806. The lower jaw member 806 may contain glue in the inner portion 808. The glue may be nonconductive. The embossed insert 803 may be plastic and/or nonconductive. A shim 809 may be used to adjust the position of the embossed insert 803 in the inner portion 808 of the lower jaw member 806. The shim 809 may have non-uniform thickness, such as the stepped configuration shown in FIG. 8A.

As illustrated in FIG. 8A, the shim 809 may be provided between the upper and lower jaw members 806, 807 along the upper electrode 810 of the upper jaw member 807. When the upper and lower jaw members 806, 807 are pushed toward each other, the shim 809 may push down on the member assembly 802 unevenly. The embossed insert 803 may be pushed into the glue contained in the inner portion 808 of the lower jaw member 806. The position of the embossed insert 803 may be adjusted per the shim geometry.

Then, as illustrated in FIG. 8B, the shim 809 may be removed from between the upper and jaw members 806, 807. The glue may be dried. The drying may be by a method and/or under a condition that may be recognized by one skilled in the art. For example, the glue may be cured at the room temperature or by heating. The glue may set and solidify such that the embossed insert 803 may be affixed in the glue at the position set by the shim 809. An electrically conductive gap setting member 811, such as a metal pin, is located on one of the upper or lower jaw members 806, 807, such as the lower jaw member 806 as shown herein, at the distal end 812 of the end effector 800 to define the non-uniform gap 813 between the upper and lower jaw members 806, 807 in the closed position. The gap setting member 811 may protrude through an opening 814 defined by the lower electrode 805. There may be a space between the inner periphery of the opening 814 and the outer periphery of the gap setting member 811 such that the gap setting member 811 may not contact any electrically conductive portion of the lower electrode 805. Although the opening 814 is shown to have a substantially round shape, the opening 814 can have any shape as long as the inner periphery of the opening 814 may not contact the outer periphery of the gap setting member 811.

In one aspect, the electrically insulative tissue engaging member assembly 802 is made of an electrically insulative material as described generally hereinabove and particularly in connection with FIGS. 3 and 5. For example, the tissue engaging member assembly 802 may be made of electrically insulative material such as a polymer and, more specifically, can be an electrically insulative material (e.g., polyimide, polyester, fluorocarbon, or any polymeric material, or any combinations thereof). The tissue engaging member assembly 802 may be generally attached to the tissue contacting side of the jaw member 806. In one aspect, the tissue engaging member assembly 802 may comprise a nonstick coating or may be made of a nonstick material such as TEFLON. Any nonstick material or nonstick surface finish may be suitable to prevent tissue from sticking to the electrically conductive portion of the upper electrode 810.

In one aspect, gap setting member 811 can be made of a metal or metal alloy and preferably can be made of steel, such as medical grade stainless steel, for example, suitable for setting a non-uniform gap 813 between the electrodes of the upper and lower jaw members 806, 807. In one aspect, the electrically conductive gap setting member 811 is an electrically conductive metal pin made of a stiff incompressible material having a high tensile strength and suitable for setting a non-uniform gap 813 between the upper and lower electrodes 810, 805. In one aspect, the gap setting member 811 is made of an electrically conductive stiff incompressible material having a high tensile strength as described generally hereinabove and particularly in connection with FIGS. 3 and 5. For example, the gap setting member 811 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

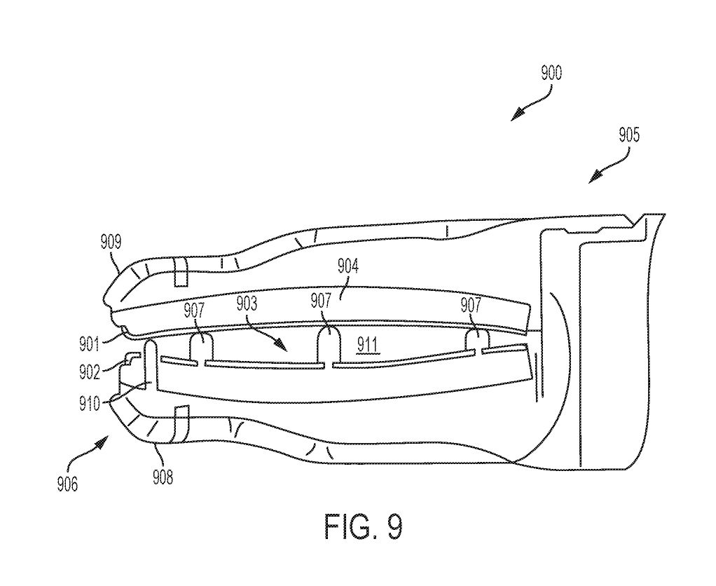

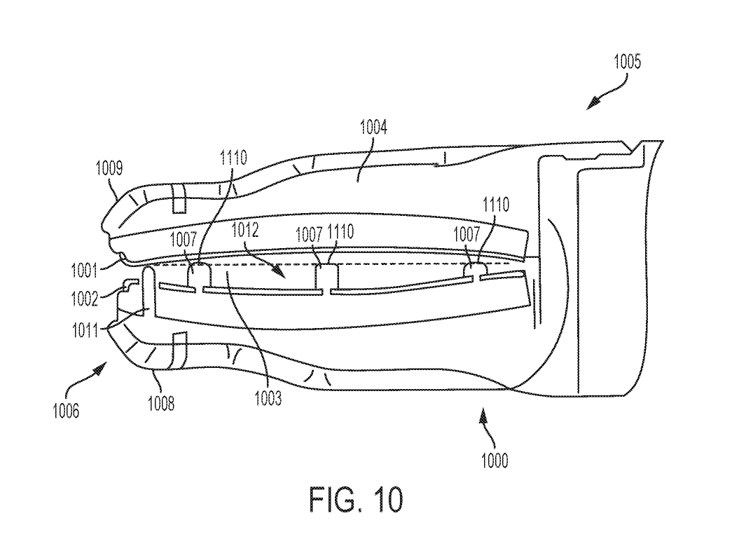

FIGS. 9 and 10 illustrate end effectors 900, 1000 comprising distal electrically conductive gap setting members 910, 1111, and electrically insulative tissue engaging members 907, 1007, according to various aspects of the present disclosure. In some aspects of the present disclosure, the electrodes may be cambered as illustrated in FIG. 9 and FIG. 10. The cambered upper and lower electrodes 901-902, 1001-1002 may form a non-uniform gap 903, 1003 therebetween with larger gap distances in the middle portion 904, 1004 of the upper and lower electrodes 901-902, 1001-1002 and smaller gap distances closer to the distal end 906, 1006 and proximal ends 905, 1005 of the end effector 900, 1000. The electrically insulative tissue engaging members 907, 1007 provided therein may have the same or different heights. The tissue engaging members 907, 1007 may or may not contact either of the opposing upper or lower electrode 901, 1001. In either configuration, however, the non-uniform gap 903 is set by a gap setting member 910 and not by the tissue engaging members 907, 1007.

In FIG. 9, the electrically insulative tissue engaging members 907 provided in the lower jaw member 908 may be configured to contact the upper electrode 901. The electrically conductive gap setting member 910 is located in one of the jaw members 908, 909, such as the lower jaw member 908, to maintain a non-uniform gap 903 distance between the upper and lower electrodes 901, 902 at the distal end 906 of the end effector 900 when the upper and lower jaw members 908, 909 are in the closed position.

In one aspect, the electrically insulative tissue engaging members 907 are made of an electrically insulative material as described generally hereinabove and particularly in connection with FIGS. 3 and 5. For example, the tissue engaging members 907 may be made of electrically insulative material such as a polymer and, more specifically, can be an electrically insulative material (e.g., polyimide, polyester, fluorocarbon, or any polymeric material, or any combinations thereof). The tissue engaging members 907 generally may be attached to the tissue contacting side of the lower jaw member 908. In one aspect, the tissue engaging members 907 may comprise a nonstick coating or may be made of a nonstick material such as TEFLON. Any nonstick material or nonstick surface finish may be suitable to prevent tissue from sticking to the electrically conductive portion of the upper electrode 901.

In one aspect, gap setting member 910 can be made of a metal or metal alloy and preferably can be made of steel, such as medical grade stainless steel, for example, suitable for setting a non-uniform gap 903 between the upper and lower electrodes 901, 902 of the upper and lower jaw members 908, 909. The gap setting member 910 and the tissue engaging members 907 may be adapted such that the upper and lower electrodes 901, 902 may not contact each other at the tissue grasping portion 911 of the end effector 900 and such as to avoid short-circuiting and/or undesired arcing of the end effector 900. In one aspect, the electrically conductive gap setting member 910 is an electrically conductive metal pin made of a stiff incompressible material having a high tensile strength and suitable for setting a non-uniform gap 903 between the upper and lower electrodes 901, 902. In one aspect, the gap setting member 910 is made of an electrically conductive stiff incompressible material having a high tensile strength as described generally hereinabove and particularly in connection with FIGS. 3 and 5. For example, the gap setting member 910 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

In FIG. 10, the electrically insulative tissue engaging members 1007 provided in the lower jaw member 1008 may be configured to not contact the upper electrode 1001. The tissue engaging members 1007 may be configured for tissue grasping, gripping, or otherwise manipulating. For example, the electrically insulative tissue engaging members 1007 may include tissue grasping surfaces 1010. An electrically conductive gap setting member 1011 is located in one of the upper or lower jaw members 1008, 1009 to maintain a non-uniform gap 1012 between the upper and lower electrodes 1001, 1002 such that that the upper and lower electrodes 1001, 1002 do not contact each other when the upper and lower jaw members 1008, 1009 are in the closed position.

In one aspect, the electrically insulative tissue engaging members 1007 are made of an electrically insulative material as described generally hereinabove and particularly in connection with FIGS. 3 and 5. For example, the tissue engaging members 1007 may be made of electrically insulative material such as a polymer and, more specifically, can be an electrically insulative material (e.g., polyimide, polyester, fluorocarbon, or any polymeric material, or any combinations thereof). The tissue engaging members 1007 may be generally attached to the tissue contacting side of the lower jaw member 1006. In one aspect, the tissue engaging members 1007 may comprise a nonstick coating or may be made of a nonstick material such as TEFLON. Any nonstick material or nonstick surface finish may be suitable to prevent tissue from sticking to the electrically conductive portion of the upper electrode 1001.

In one aspect, gap setting member 1011 can be made of a metal or metal alloy and preferably can be made of steel, such as medical grade stainless steel, for example, suitable for setting a non-uniform gap 1012 between the electrodes of the upper and lower jaw members 1008, 1009. In one aspect, the electrically conductive gap setting member 1011 is an electrically conductive metal pin made of a stiff incompressible material having a high tensile strength and suitable for setting a non-uniform gap 1012 between the upper and lower electrodes 1001, 1002. In one aspect, the gap setting member 1011 is made of an electrically conductive stiff incompressible material having a high tensile strength as described generally hereinabove and particularly in connection with FIGS. 3 and 5. For example, the gap setting member 1011 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

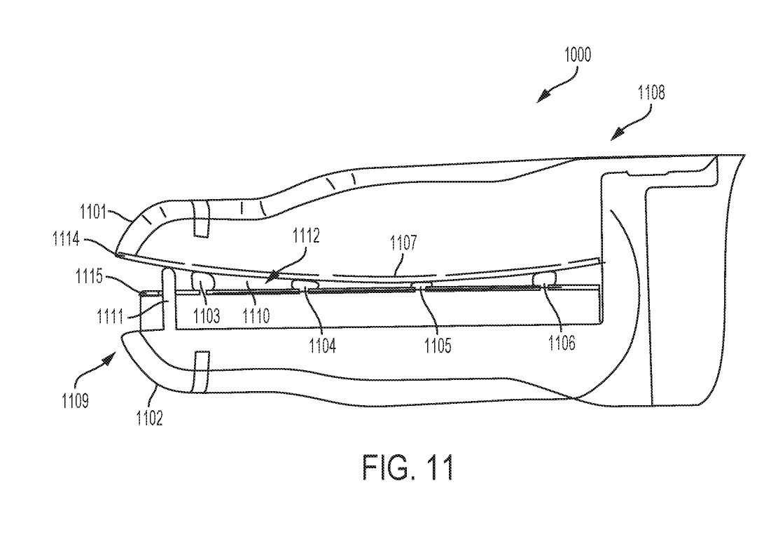

In some other aspects of the present disclosure, as illustrated in FIG. 11, one of the upper or lower jaw members 1101, 1102, such as the upper jaw member 1101, of an end effector 1100 may deflect when a force is applied on the upper jaw member 1101. The upper jaw member 1101 may be initially flat when no force is applied to it. When a force, such as a clamp force, is applied to it, the upper jaw member 1101 may move toward the lower jaw member 1102 until contacting the electrically insulative tissue engaging members 1103, 1104, 1105, 1106 provided on the lower jaw member 1102. The tissue engaging members 1103-1106 may have different heights. The tissue engaging members 1104-1105 in the middle portion 1107 of the lower jaw member 1102 may be shorter than the tissue engaging members 1103, 1106 closer to the distal end 1109 and proximal 1108 ends of the end effector 1100. Before the upper jaw member 1101 contacts any of the tissue engaging members 1103-1106, the upper jaw member 1101 may stay flat without deformation. When the upper jaw member 1101 contacts at least one of the tissue engaging members 1103-1106, such as the tissue engaging members 1103, 1106 with the most heights, and when the force is kept on the upper jaw member, the upper jaw member 1101 may start deforming. The middle portion 1107 of the upper jaw member 1101 may bend toward the lower jaw member 1102 until it contacts the tissue engaging members 1104, 1105 with smaller heights. The upper jaw member 1101 may supper deforming until it may have contacted all the tissue engaging members 1103-1106. An electrically conductive gap setting member 1111 is located at the distal end 1109 of the end effector 1100 of the lower jaw member 1102 to define a non-uniform gap 1112. The upper jaw member 1101 may contact the gap setting member 1111 first before starting to deflect. The non-uniform gap 1112 maintained by the gap setting member 1111 at the distal end 1109 may be the largest between the jaw members.

In one aspect, the electrically insulative tissue engaging members 1103-1106 are made of an electrically insulative material as described generally hereinabove and particularly in connection with FIGS. 3 and 5. For example, the tissue engaging members 1103-1106 may be made of electrically insulative material such as a polymer and, more specifically, can be an electrically insulative material (e.g., polyimide, polyester, fluorocarbon, or any polymeric material, or any combinations thereof). The tissue engaging members 1103-1106 may be generally attached to the tissue contacting side of the lower jaw member 1102. In one aspect, the tissue engaging members 1103-1106 may comprise a nonstick coating or may be made of a nonstick material such as TEFLON. Any nonstick material or nonstick surface finish may be suitable to prevent tissue from sticking to the electrically conductive portion of the upper electrode 1114.

In one aspect, gap setting member 1111 can be made of a metal or metal alloy and preferably can be made of steel, such as medical grade stainless steel, for example, suitable for setting a non-uniform gap 1112 between the upper and lower electrodes 1114, 1115 of the upper and lower jaw members 1101, 1102. In one aspect, the electrically conductive gap setting member 1111 is an electrically conductive metal pin made of a stiff incompressible material having a high tensile strength and suitable for setting a non-uniform gap 1112 between the upper and lower electrodes 1114, 1115. In one aspect, the gap setting member 1111 is made of an electrically conductive stiff incompressible material having a high tensile strength as described generally hereinabove and particularly in connection with FIGS. 3 and 5. For example, the gap setting member 1111 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

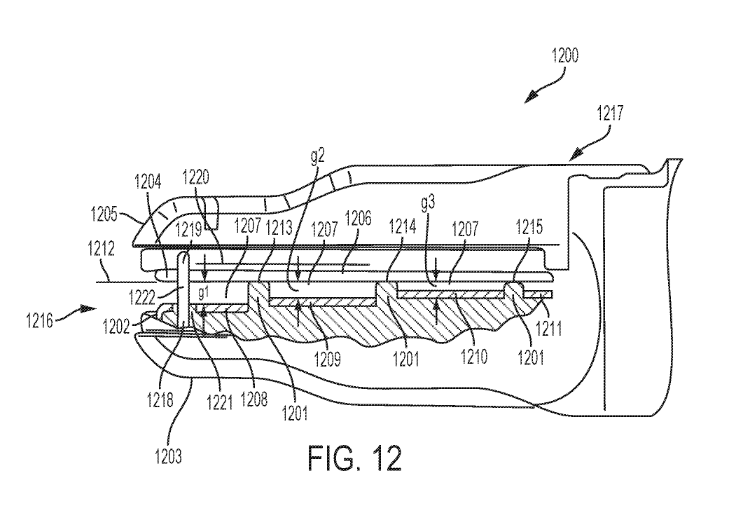

FIG. 12 shows a cross-sectional view of an end effector 1200 comprising a distal electrically conductive gap setting member 1218 and electrically insulative tissue engaging members 1201 provided on a stepped or terraced lower electrode 1202, according to one aspect of the present disclosure. The lower electrode 1202 may be provided on the lower jaw member 1203 as the lower electrode 1202. The upper electrode 1204 on the upper jaw member 1205 may be located on the same plane 1206. The lower electrode 1202 may define steps 1207 on different planes 1208, 1209, 1210, 1211. The tissue engaging members 1201 may extend from the steps 1208-1210 to the same plane 1212. When the upper and lower jaw members 1205, 1203 are in the closed position, the upper surfaces 1213-1215 of the tissue engaging members 1201 may contact the upper electrode 1204, and the planes 1206, 1212 may overlap with each other.

An electrically conductive gap setting member 1218 is located at the distal end 1216 of the end effector 1200 and is configured to form a non-uniform gap defined by g1, g2, g3 between the jaw upper and lower members 1205, 1203. The gap distances g1, g2, g3 between the upper and lower jaw members 1205, 1203 at the steps 1207 may decrease along the length of the upper and lower jaw members 1205, 1203 from the distal end 1216 to the proximal end 1217 of the end effector 1200. The gap setting member 1218 may be a metal pin provided on one of the upper and lower jaw members 1205, 1203. The gap setting member 1218 may protrude through an opening 1221 in the lower electrode 1202. There may be a space between the inner periphery of the opening 1221 and the outer periphery of the gap setting member 1218 such that the gap setting member 1218 may not contact any electrically conductive portion of the lower electrode 1202. Although the opening 1221 is shown to have a substantially round shape, the opening 1221 can have any shape as long as the inner periphery of the opening 1221 may not contact the outer periphery of the gap setting member 1218. The gap setting member 1218 may extend from the lower jaw member 1203 toward the upper jaw member 1205. The gap setting member 1218 may contact a non-electrically conductive portion 1222 in the upper electrode 1204 or protrude through a opening 1219 in the upper electrode 1204 into the inner portion 1220 of the upper jaw member 1205. The inner portion 1220 of the upper jaw member 1205 may be non-electrically conductive.

In one aspect, the electrically insulative tissue engaging members 1201 are made of an electrically insulative material as described generally hereinabove and particularly in connection with FIGS. 3 and 5. For example, the tissue engaging members 1201 may be made of electrically insulative material such as a polymer and, more specifically, can be an electrically insulative material (e.g., polyimide, polyester, fluorocarbon, or any polymeric material, or any combinations thereof). The tissue engaging members 1201 may be generally attached to the tissue contacting side of the lower jaw member 1203. In one aspect, the tissue engaging members 1007 may comprise a nonstick coating or may be made of a nonstick material such as TEFLON. Any nonstick material or nonstick surface finish may be suitable to prevent tissue from sticking to the electrically conductive portion of the upper electrode 1204.

In one aspect, gap setting member 1218 can be made of a metal or metal alloy and preferably can be made of steel, such as medical grade stainless steel, for example, suitable for setting a non-uniform gap between the upper and lower electrodes 1204, 1202 of the upper and lower jaw members 1205, 1203. In one aspect, the electrically conductive gap setting member 1218 is an electrically conductive metal pin made of a stiff incompressible material having a high tensile strength and suitable for setting gap distances g1, g2, g3 (e.g., non-uniform gap) between the upper and lower electrodes 1204, 1202. In one aspect, the gap setting member 1218 is made of an electrically conductive stiff incompressible material having a high tensile strength as described generally hereinabove and particularly in connection with FIGS. 3 and 5. For example, the gap setting member 1218 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

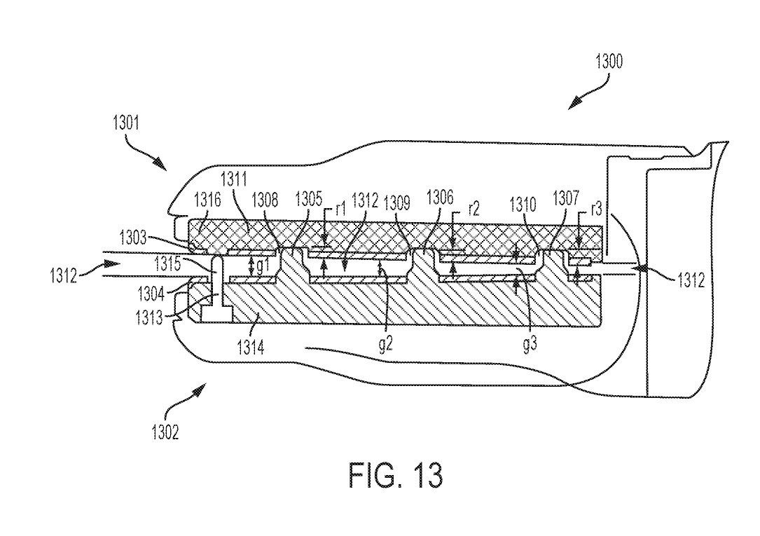

FIG. 13 shows a cross-sectional view of an end effector 1300 comprising a distal electrically conductive gap setting member 1313 and electrically insulative tissue engaging members 1305, 1306, 1307, according to one aspect of the present disclosure. The end effector 1300 may include an upper jaw member 1301 and a lower jaw member 1302. A upper electrode 1303 may be provided on a lower surface of the upper jaw member 1301. A lower electrode 1304 may be provided on an upper surface of the lower jaw member 1302. The tissue engaging members 1305-1307 may be provided on the lower jaw member 1302. Recesses 1308, 1309, 1310 may be provided on or defined by the upper electrode 1303 corresponding to the tissue engaging members 1305-1307. The tissue engaging members 1305-1307 and the recesses 1308-1310 may be configured such that upper portions of the members 1305-1307 may be received in the recesses 1308-1310 without contacting the upper electrode 1303. The tissue engaging members 1305-1307 may contact an inner portion 1311 of the upper jaw member 1301. The inner portion 1311 of the upper jaw member 1301 may be nonconductive. The tissue engaging members 1305-1307 may have the same height. The recesses 1308-1310 may have different depths r1, r2, r3. The tissue engaging members 1305-1307 and the corresponding recesses 1308-1310 may be configured to form a non-uniform gap 1312 with different heights g1-g3 between the upper and lower jaw members 1301, 1302.

In one aspect, the electrically insulative tissue engaging members 1305-1307 are made of an electrically insulative material as described generally hereinabove and particularly in connection with FIGS. 3 and 5. For example, the tissue engaging members 1305-1307 may be made of electrically insulative material such as a polymer and, more specifically, can be an electrically insulative material (e.g., polyimide, polyester, fluorocarbon, or any polymeric material, or any combinations thereof). The tissue engaging members 1305-1307 may be generally attached to the tissue contacting side of the jaw lower member 1302. In one aspect, the tissue engaging members 1305-1307 may comprise a nonstick coating or may be made of a nonstick material such as TEFLON. Any nonstick material or nonstick surface finish may be suitable to prevent tissue from sticking to the electrically conductive portion of the upper electrode 1303.

An electrically conductive gap setting member 1313 is located on one of the jaws, such as the lower jaw member 1302, at the distal end of the end effector 1300. The gap setting member 1313 is electrically conductive. The gap setting member 1313 may be a metal pin. The gap setting member 1313 defines the non-uniform gap 1312. The gap setting member 1313 may be affixed on and extend from a lower 1314 of the lower jaw member 1302 through an opening 1315 defined by the lower electrode 1304. The gap setting member 1313 may not contact the upper electrode 1303. As shown here, an opening 1316 may be defined in the upper electrode 1303, and the inner portion 1311 of the upper jaw member 1301 may be exposed. The gap setting member 1313 may contact the inner portion 1311 of the upper jaw member 1301, but not the upper electrode 1303. The inner portion 1311 of the upper jaw member 1301 may be non-electrically conductive or connected to ground.

In one aspect, gap setting member 1313 can be made of a metal or metal alloy and preferably can be made of steel, such as medical grade stainless steel, for example, suitable for setting a non-uniform gap 1312 between the upper and lower electrodes 1303, 1304 of the upper and lower jaw members 1301, 1302. In one aspect, the electrically conductive gap setting member 11313 is an electrically conductive metal pin made of a stiff incompressible material having a high tensile strength and suitable for setting gap distances g1, g2, g3 (e.g., non-uniform gap) between the upper and lower electrodes 1303, 1304. In one aspect, the gap setting member 1313 is made of an electrically conductive stiff incompressible material having a high tensile strength as described generally hereinabove and particularly in connection with FIGS. 3 and 5. For example, the gap setting member 1313 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

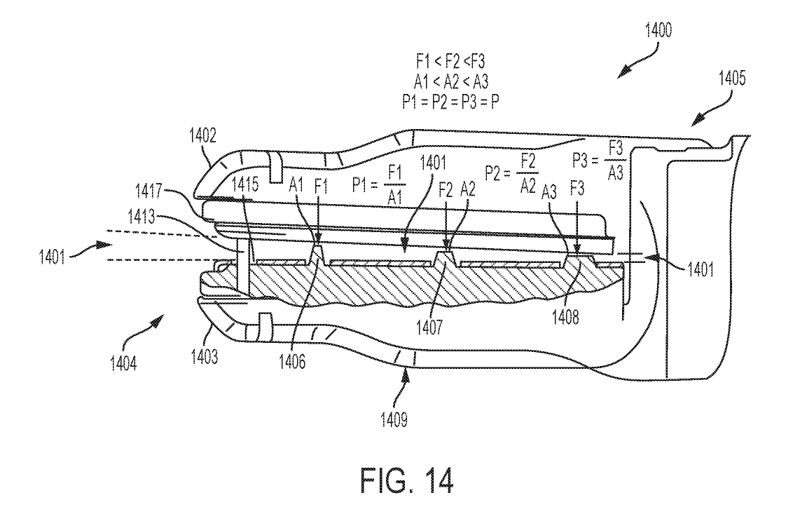

FIG. 14 shows an end effector 1400 comprising a distal electrically conductive gap setting member 1413 and electrically insulative tissue engaging members 1406, 1407, 1408, according to one aspect of the present disclosure. In some aspects of the present disclosure, the electrically insulative tissue engaging members may be compliant and configured to deform uniformly under a specified pressure. As illustrated in FIG. 14, a non-uniform gap 1401 may be made between the upper and lower jaw members 1402, 1403. The gap distance may decrease along the length of the jaw members 1402-1403 from the distal end 1404 toward the proximal end 1405 of the end effector 1400. The tissue engaging members 1406-1408 may be configured to have different upper surface areas A1, A2, A3. A1 may be smaller than A2, and A2 may be smaller than A3. When the upper jaw member 1402 pushes down on the tissue engaging members 1406-1408, the forces F1, F2, F3 applied on the tissue engaging members 1406-1408 may increase along the length of the upper and lower jaw members 1402-1403 from the distal end 1404 toward the proximal end 1405 of the end effector 1400. The force F1 applied to the tissue engaging member 1406 closer to the distal end may be smaller than the force F2 applied to the tissue engaging member 1407 in the middle portion 1409 of the end effector 1400. The force F2 applied to the electrically insulative member 1407 the middle portion 1409 of the end effector 1400 may be smaller than the force F3 applied to the tissue engaging member 1408 closer to the proximal end 1405 of the end effector 1400. The upper surface areas A1-A3 of the tissue engaging members 1406-1408 may be configured such that the pressures P1-P3 on the tissue engaging members 1406-1408 may be the same. The tissue engaging members 1406-1408 may deform uniformly under the pressures P1-P3. The pressures P1-P3 may be a specified pressure P.

In one aspect, the electrically insulative tissue engaging members 1406-1408 are made of an electrically insulative material as described generally hereinabove and particularly in connection with FIGS. 3 and 5. For example, the tissue engaging members 1406-1408 may be made of electrically insulative material such as a polymer and, more specifically, can be an electrically insulative material (e.g., polyimide, polyester, fluorocarbon, or any polymeric material, or any combinations thereof). The tissue engaging members 1406-1408 may be generally attached to the tissue contacting side of the jaw member 1403. In one aspect, the tissue engaging members 1406-1408 may comprise a nonstick coating or may be made of a nonstick material such as TEFLON. Any nonstick material or nonstick surface finish may be suitable to prevent tissue from sticking to the electrically conductive portion of the upper electrode 1417.

An electrically conductive gap setting member 1413 is located on one of the upper or lower jaw members 1402, 1403, such as the lower jaw member 1403, at the distal end 1404 of the end effector 1400. The gap setting member 1413 is electrically conductive. The gap setting member 1413 may be a metal pin. The gap setting member 1413 defines the non-uniform gap 1401. The gap setting member 1401 may be affixed on and extend from the lower jaw member 1403 through an opening 1415 defined the lower electrode 1416. In one aspect, gap setting member 1413 can be made of a metal or metal alloy and preferably can be made of steel, such as medical grade stainless steel, for example, suitable for setting a non-uniform gap 1401 between the upper and lower electrodes 1416, 1417 of the upper and lower jaw members 1402, 1403. In one aspect, the electrically conductive gap setting member 1413 is an electrically conductive metal pin made of a stiff incompressible material having a high tensile strength and suitable for setting a non-uniform gap 1401 between the upper and lower electrodes 1417, 1416. In one aspect, the gap setting member 1413 is made of an electrically conductive stiff incompressible material having a high tensile strength as described generally hereinabove and particularly in connection with FIGS. 3 and 5. For example, the gap setting member 1413 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

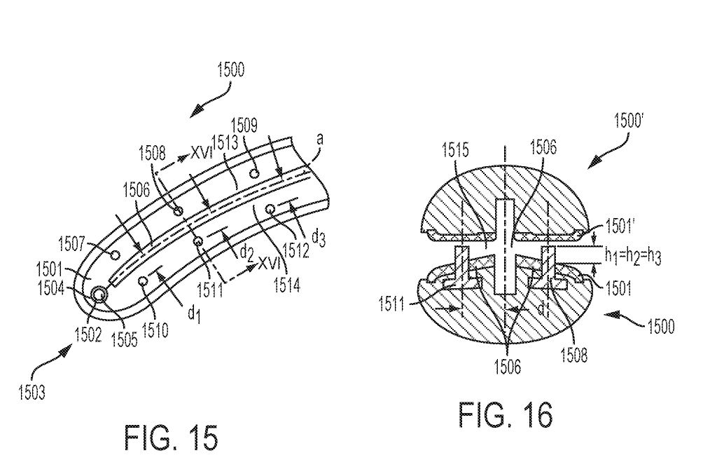

FIG. 15 shows a plan view of one aspect of a jaw member 1500 comprising a distal electrically conductive gap setting member 1502 and electrically insulative tissue engaging members 1507-1512 evenly positioned along a width of the jaw member 1500, according to one aspect of the present disclosure. An electrode 1501 may be provided on one surface of the jaw member 1500. An electrically conductive gap setting member 1502 is located at the distal end 1503 of the jaw member 1500. The gap setting member 1502 may protrude through an opening 1504 defined by the electrode 1501. There may be a space 1505 between the inner periphery of the opening 1504 and the outer periphery of the gap setting member 1502 such that the gap setting member 1502 may not contact any electrically conductive portion of the electrode 1501. Although the opening 1504 is shown to have a substantially round shape, the opening 1504 can have any shape as long as the inner periphery of the opening 1504 does not contact the outer periphery of the gap setting member 1502.

A knife channel 1506 may be provided in the interior, such as the middle, of the jaw member 1500. A cutting member, not shown here, may be provided in the knife channel 1506 for cutting tissue after the tissue has been sealed using electrosurgical energy.

At least one electrically insulative tissue engaging member 1507-1512 is provided on the electrode 1501. The electrically insulative tissue engaging members 1507-1512 are located on two sides 1513, 1514 of the electrode 1501 along the knife channel 1506. The tissue engaging members 1507-1512 may have the same or different shapes and may be spaced evenly or unevenly. The distances between the tissue engaging members 1507-1512 and the knife channel 1506 may be the same or different. As shown here, the distances d₁, d₂, and d₃ between the tissue engaging members 1507-1512 and a longitudinal center axis a along the knife channel 1506 may be the same.

In one aspect, the electrically insulative tissue engaging members 1507-1512 are made of an electrically insulative material as described generally hereinabove and particularly in connection with FIGS. 3 and 5. For example, the tissue engaging members 1507-1512 may be made of electrically insulative material such as a polymer and, more specifically, can be an electrically insulative material (e.g., polyimide, polyester, fluorocarbon, or any polymeric material, or any combinations thereof). The tissue engaging members 1507-1512 may be generally attached to the tissue contacting side of the jaw member 1500. In one aspect, the tissue engaging members 1507-1512 may comprise a nonstick coating or may be made of a nonstick material such as TEFLON. Any nonstick material or nonstick surface finish may be suitable to prevent tissue from sticking to the electrically conductive portion of the upper electrode (not shown).

In one aspect, the electrically conductive gap setting member 1502 is an electrically conductive metal pin made of a stiff incompressible material having a high tensile strength and suitable for setting a gap between the upper electrode (not shown) and the electrode 1501. In one aspect, the gap setting member 1502 is made of an electrically conductive stiff incompressible material having a high tensile strength as described generally hereinabove and particularly in connection with FIGS. 3 and 5. For example, the gap setting member 1502 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

FIG. 16 shows the XVI-XVI sectional view of one aspect of an end effector with the jaw member 1500 of FIG. 15, according to one aspect of the present disclosure. At least one of the upper or lower electrodes 1501, 1501′ define a taper 1516 that forms a non-uniform gap 1515 between the upper and lower electrodes 1501, 1501′ of the upper and lower jaw members 1500, 1500′. The non-uniform gap 1515 between the upper or lower electrodes 1501, 1501′ defined by the taper 1516 is narrowest along the central portion of the upper and lower jaw members 1500, 1500′, for example, along the knife channel 1506, and gradually increases laterally toward the outside edges of the upper and lower jaw members 1500, 1500′. The electrically insulative tissue engaging members 1507-1512 are disposed along the length of the lower electrode 1501, such that the opposing upper electrode 1501′ does not contact the tissue engaging members 1507-1512.

With reference to FIGS. 15 and 16, a non-uniform but consistent spacing between the upper and lower electrodes 1501, 1501′ of the upper and lower jaw members 1500, 1500′ is set by the electrically conductive gap setting member 1502. The tissue engaging members 1507-1512 may be positioned evenly, as shown in FIG. 15, having the same distance “d” to the knife channel 1506, and be of uniform height. As shown here, the heights h₁, h₂, h₃ of the tissue engaging members 1507-1509 may be the same. In one aspect, gap setting member 1502, as shown in FIG. 15, can be made of a metal or metal alloy and preferably can be made of steel, such as medical grade stainless steel, for example, suitable for setting a gap between the upper and lower electrodes 1501′, 1501 of the upper and lower jaw members 1500′, 1500.

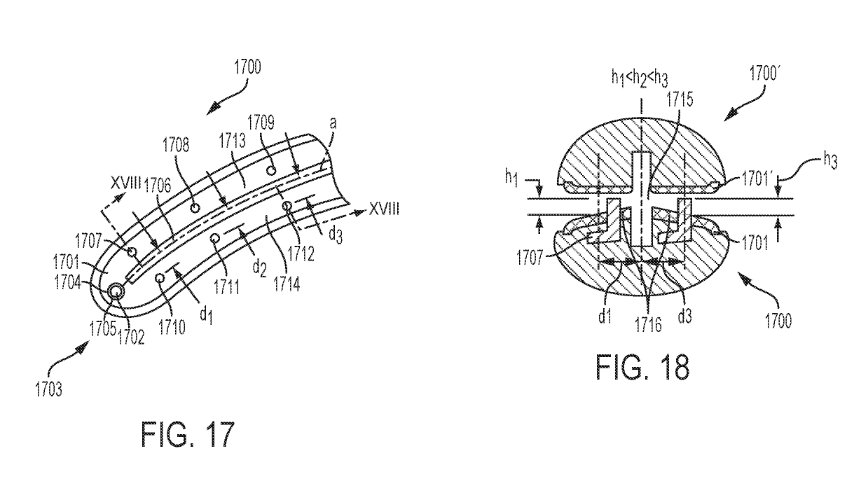

Alternatively, FIG. 17 shows a plan view of one aspect of a jaw member 1700 comprising a distal electrically conductive gap setting member 1702 and electrically insulative tissue engaging members 1707-1712 oriented in a staggered uneven position and of non-uniform height, for example, according to one aspect of the present disclosure. An electrode 1701 is provided on one surface of the jaw member 1700. The gap setting member 1702 is located at the distal end 1703 of the jaw member 1700. The gap setting member 1702 protrudes through an opening 1704 defined by the electrode 1701. A space 1705 is provided between the inner periphery of the opening 1704 and the outer periphery of the gap setting member 1702 such that the member 1702 does not contact any electrically conductive portion of the electrode 1701. Although the opening 1704 is shown to have a substantially round shape, the opening 1704 can have any shape as long as the inner periphery of the opening 1704 may not contact the outer periphery of the gap setting member 1702.

A knife channel 1706 may be provided in the interior, such as the middle, of the jaw member 1700. A cutting member, not shown here, may be provided in the knife channel 1706 for cutting tissue after the tissue has been sealed using electrosurgical energy.

At least one electrically insulative member may be provided on the electrode 1701. The tissue engaging members 1707-1712 can be provided on the electrode 1701 on each side 1713, 1714 of and along the knife channel 1706. The members 1707-1712 may have the same or different shapes and may be spaced evenly or unevenly. The distances between the tissue engaging members 1707-1712 and the knife channel 1706 may be the same or different. As shown here, the distances d₁, d₂, d₃ between the tissue engaging members 1710-1712 and a longitudinal center axis a along the knife channel 1706 may be the same.

In one aspect, the electrically insulative tissue engaging members 1707-1712 are made of an electrically insulative material as described generally hereinabove and particularly in connection with FIGS. 3 and 5. For example, the tissue engaging members 1707-1712 may be made of electrically insulative material such as a polymer and, more specifically, can be an electrically insulative material (e.g., polyimide, polyester, fluorocarbon, or any polymeric material, or any combinations thereof). The tissue engaging members 1707-1712 may be generally attached to the tissue contacting side of the jaw member 1700. In one aspect, the tissue engaging members 1707-1712 may comprise a nonstick coating or may be made of a nonstick material such as TEFLON. Any nonstick material or nonstick surface finish may be suitable to prevent tissue from sticking to the electrically conductive portion of the upper electrode (not shown).

In one aspect, the electrically conductive gap setting member 1702 is an electrically conductive metal pin made of a stiff incompressible material having a high tensile strength and suitable for setting a gap between the upper electrode (not shown) and the lower electrode 1701. In one aspect, the gap setting member 1702 is made of an electrically conductive stiff incompressible material having a high tensile strength as described generally hereinabove and particularly in connection with FIGS. 3 and 5. For example, the gap setting member 1702 can be made of metal or metal alloy and preferably can be made of steel, such as medical grade biocompatible stainless steel, for example, as well as electrically conductive components such as copper, silver, aluminum, tungsten, nickel, or any other electrically conductive materials that may occur to those skilled in the art.

FIG. 18 shows the XVIII-XVIII sectional view of one aspect of an end effector with the jaw member of FIG. 17, according to one aspect of the present disclosure. At least one of the upper or lower electrodes 1701′, 1701 define a taper 1716 that forms a non-uniform gap 1715 between the upper and lower jaw members 1700, 1700′. The non-uniform gap 1715 between the upper or lower electrodes 1701′, 1701 defined by the taper 1716 is narrowest along the central portion of the upper and lower jaw members 1700′, 1700, for example, along the knife channel 1706, and gradually increases laterally toward the outside edges of the upper and lower jaw members 1700′, 1700. The tissue engaging members 1707-1712 are disposed along the length of the lower electrode 1701, such that the opposing upper electrode 1701′ does not contact the tissue engaging members 1707-1712.

With reference to FIGS. 17 and 18, a non-uniform but consistent spacing between the upper and lower jaw members 1700′, 1700 is provided between the jaw members 1300′, 1300 both laterally and longitudinally. However, different from the aspects of FIGS. 15 and 16, the tissue engaging members 1707-1712 may be positioned unevenly, as shown in FIG. 17, and be of non-uniform heights. The tissue engaging member 1707 and its corresponding tissue engaging member 1710 across the knife channel 1706 along a width of the lower jaw member 1700 may have the same distance d₁ from the longitudinal center axis a along the knife channel 1706 and the same height h₁. Similarly, the tissue engaging member 1708 and its corresponding tissue engaging member 1711 across the knife channel 1706 along a width of the lower jaw member 1700 may have the same distance d₂ from the longitudinal center axis a along the knife channel 1706 and the same height h₂ (not shown in the figures), and the tissue engaging member 1709 and its corresponding tissue engaging member 1712 across the knife channel 1706 along a width of the jaw member 1700 may have the same distance d₃ from the longitudinal center axis a along the knife channel 1706 and the same height h₃. As shown here, the heights h₁, h₂, h₃ may be different, for example, h₁<h₂<h₃. In one aspect, the gap setting member 1702, as shown in FIG. 17, can be made of a metal or metal alloy and preferably can be made of steel, such as medical grade stainless steel, for example, suitable for setting a gap between the upper and lower electrodes 1701′, 1701 of the upper and lower jaw members 1700′, 1700.

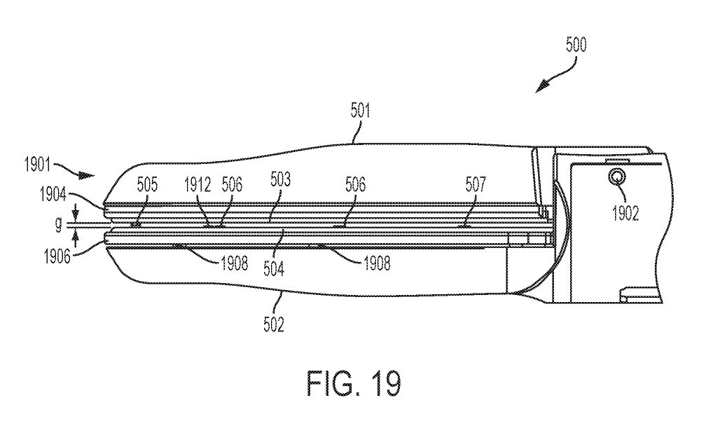

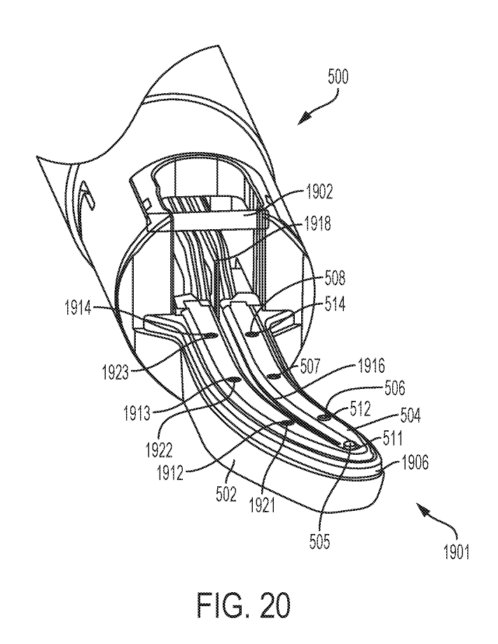

FIG. 19 is a side elevational view of the end effector 500 of FIG. 5, according to one aspect of the present disclosure. The end effector 500 comprises an upper jaw member 501 and a lower jaw member 502, where the upper jaw member 501 is pivotally movable between open and closed positions about a pivot 1902. As shown in FIG. 19, the upper jaw member 501 is in a closed position. The end effector 500 comprises an upper electrode 503 and a lower electrode 504. The upper electrode 503 is electrically isolated from the upper jaw member 501 by an electrically insulative element 1904 and the lower electrode 504 is electrically isolated from the lower jaw member 502 by another electrically insulative element 1906. The lower electrically insulative element 1906 and the lower electrode 504 are supported on the lower jaw member 502 by support members 1908. An electrically conductive gap setting member 505 is located on the distal end 1901 of the lower jaw member 502 and protrudes through an opening 1920 (see FIGS. 20, 23, 24) defined by the lower electrode 504 to electrically isolate the gap setting member 505 from the lower electrode 504. The gap setting member 505 sets a gap “g” between the upper and the lower electrodes 503, 504. Electrically insulative tissue engaging members 506-508, 1912 are disposed on the lower jaw member 502. The tissue engaging members 506-508, 1912 protrude through openings 1921, 512, 513, 514 (see FIGS. 20, 23, 24) defined by the lower electrode 504. The tissue engaging members 506-508, 1912 tightly or loosely fit through the openings.

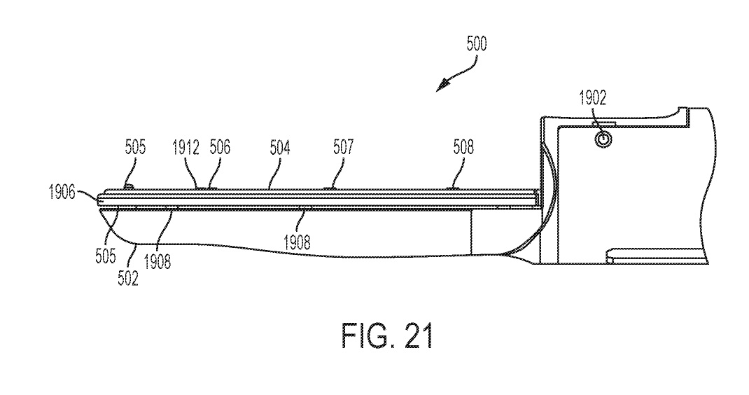

FIG. 20 shows a perspective view of the lower jaw member 502 of FIG. 19, according to one aspect of the present disclosure and FIG. 21 shows a side elevational view of the lower jaw member 502 of FIG. 19, according to one aspect of the present disclosure. With reference to FIGS. 20 and 21, the lower electrode 504 may be provided on an upper surface of the lower jaw member 502. The gap setting member 505 may be a metal pin provided on the upper face of the lower jaw member 502. The gap setting member 505 may protrude through an opening 511 defined by the electrode 504 without contacting the electrode 504. The electrically insulative tissue engaging members 506-508, 1912-1914 may be provided on the lower jaw member 502. The tissue engaging members 506-508 protrude through openings 512, 513, 514 defined by the lower electrode 504 and the tissue engaging members 1912-1914 protrude through openings 1921, 1922, 1923 also defined by the lower electrode 504.

Also shown in FIGS. 20 and 21 is a knife channel 1916 to slidably reciprocate a knife 1918 therealong. One set of the electrically insulative tissue engaging members 506-508 are located on one side of the knife channel 1916 and another set of the electrically insulative tissue engaging members 1912-1914 are located on the other side of the knife channel 1916. Also, shown in this view is the pivot 1902 about which the upper jaw member 501 (FIG. 19) pivots. As shown in FIG. 21, the gap setting member 505 is supported by the lower jaw member 502.

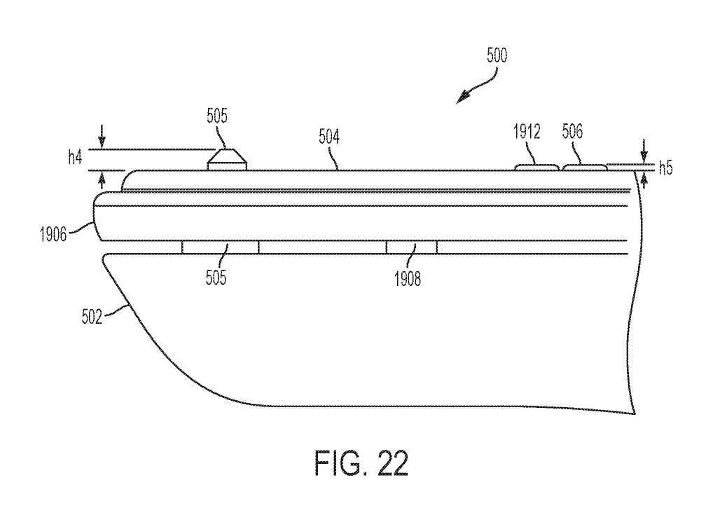

FIG. 22 shows a detail view of the distal end 1901 of the lower jaw member 502 of FIG. 21, according to one aspect of the present disclosure. As shown, the electrically conductive gap setting member 505 protrudes through the lower electrode 504 and the lower electrically insulative element 1906 and is supported by the lower jaw member 502. The lower electrode 504 is supported by the electrically insulative element 1906, which is supported by support members 1908. This view shows the difference in height “h4” of the gap setting member 505 and the “h5” of the electrically insulative tissue engaging members 1912, 506. As previously discussed, the h5<h4 such that the gap setting member 505 sets the gap “g” between the upper and lower electrodes 503, 504.

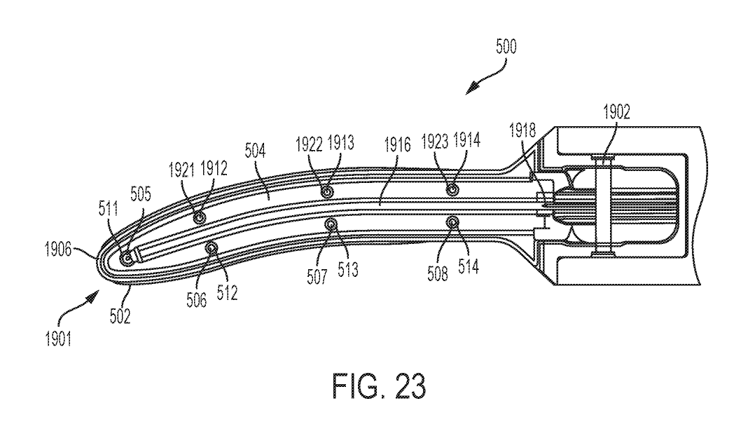

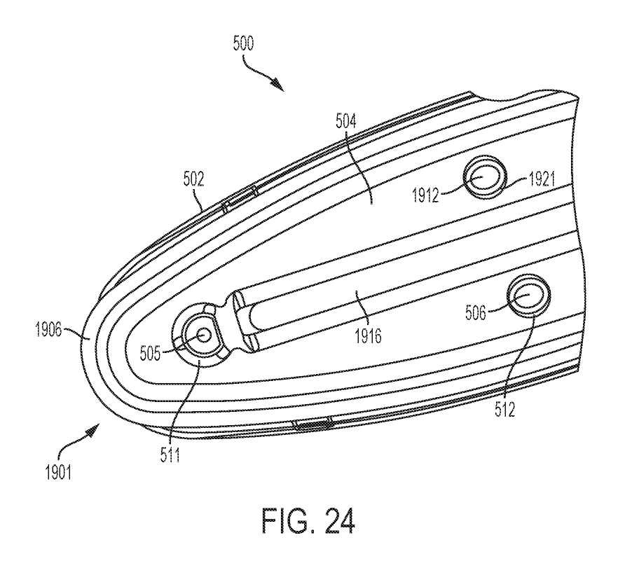

FIG. 23 shows a plan view of the lower jaw member 502 of FIG. 19, according to one aspect of the present disclosure. FIG. 24 shows a detail plan view of the distal end 1901 of the lower jaw member 302 of FIG. 23, according to one aspect of the present disclosure. With reference now to FIGS. 23 and 24, the gap setting member 505 protrudes through the opening 1920 defined by the lower electrode 504 without contacting the lower electrode 504. The electrically insulative tissue engaging members 506-508, 1912-1914 are disposed on either side of the knife channel 1916.

It will be appreciated that the terms “proximal” and “distal” are used throughout the specification with reference to a clinician manipulating one end of an instrument used to treat a patient. The term “proximal” refers to the portion of the instrument closest to the clinician and the term “distal” refers to the portion located furthest from the clinician. It will further be appreciated that for conciseness and clarity, spatial terms such as “vertical,” “horizontal,” “up,” “down,” “upper,” “lower,” “top,” or “bottom” may be used herein with respect to the illustrated aspects. However, surgical instruments may be used in many orientations and positions, and these terms are not intended to be limiting or absolute.

Various aspects of surgical instruments and robotic surgical systems are described herein. It will be understood by those skilled in the art that the various aspects described herein may be used with the described surgical instruments and robotic surgical systems. The descriptions are provided for example only, and those skilled in the art will understand that the disclosed examples are not limited to only the devices disclosed herein, but may be used with any compatible surgical instrument or robotic surgical system.

While various aspects herein have been illustrated by description of several aspects, and while the illustrative aspects have been described in considerable detail, it is not the intention of the applicant to restrict or in any way limit the scope of the appended claims to such detail. Additional advantages and modifications may readily appear to those skilled in the art. For example, it is generally accepted that endoscopic procedures are more common than laparoscopic procedures. Accordingly, the present invention has been discussed in terms of endoscopic procedures and apparatus. However, use herein of terms such as “endoscopic”, should not be construed to limit the present invention to an instrument for use only in conjunction with an endoscopic tube (e.g., trocar). On the contrary, it is believed that the present invention may find use in any procedure where access is limited to a small incision, including but not limited to laparoscopic procedures, as well as open procedures.

While the examples herein are described mainly in the context of electrosurgical instruments, it should be understood that the teachings herein may be readily applied to a variety of other types of medical instruments. By way of example only, the teachings herein may be readily applied to tissue graspers, tissue retrieval pouch deploying instruments, surgical staplers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

Aspects of the devices disclosed herein can be designed to be disposed of after a single use, or they can be designed to be used multiple times. Aspects may, in either or both cases, be reconditioned for reuse after at least one use. Reconditioning may include any combination of the steps of disassembly of the device, followed by cleaning or replacement of particular pieces, and subsequent reassembly. In particular, aspects of the device may be disassembled, and any number of the particular pieces or parts of the device may be selectively replaced or removed in any combination. Upon cleaning and/or replacement of particular parts, aspects of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, aspects described herein may be processed before surgery. First, a new or used instrument may be obtained and, if necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device also may be sterilized using any other technique known in the art, including but not limited to beta or gamma radiation, ethylene oxide, or steam.

Having shown and described various aspects of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, aspects, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It is worthy to note that any reference to “one aspect,” “an aspect,” “one aspect,” or “an aspect” means that a particular feature, structure, or characteristic described in connection with the aspect is included in at least one aspect. Thus, appearances of the phrases “in one aspect,” “in an aspect,” “in one aspect,” or “in an aspect” in various places throughout the specification are not necessarily all referring to the same aspect. Furthermore, the particular features, structures or characteristics may be combined in any suitable manner in one or more aspects.

Although various aspects have been described herein, many modifications, variations, substitutions, changes, and equivalents to those aspects may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed aspects. The following claims are intended to cover all such modification and variations.

Some or all of the aspects described herein may generally comprise technologies for flexible circuits for electrosurgical instruments, or otherwise according to technologies described herein.

All of the above-mentioned U.S. patents, U.S. patent application publications, U.S. patent applications, foreign patents, foreign patent applications, non-patent publications referred to in this specification and/or listed in any Application Data Sheet, or any other disclosure material are incorporated herein by reference, to the extent not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will be incorporated only to the extent that no conflict arises between that incorporated material and the existing disclosure material.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken as limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different or other components. It is to be understood that such depicted architectures are merely exemplary, and that, in fact, many other architectures may be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

Some aspects may be described using the expression “coupled” and “connected” along with their derivatives. It should be understood that these terms are not intended as synonyms for each other. For example, some aspects may be described using the term “connected” to indicate that two or more members are in direct physical or electrical contact with each other. In another example, some aspects may be described using the term “coupled” to indicate that two or more members are in direct physical or electrical contact. The term “coupled,” however, also may mean that two or more members are not in direct contact with each other, but yet still co-operate or interact with each other.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Further, a sale of a system or method may likewise occur in a territory even if components of the system or method are located and/or used outside the territory. Further, implementation of at least part of a system for performing a method in one territory does not preclude use of the system in another territory.

Although various aspects have been described herein, many modifications, variations, substitutions, changes, and equivalents to those aspects may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed aspects. The following claims are intended to cover all such modification and variations.

In summary, numerous benefits have been described that result from employing the concepts described herein. The foregoing description of the one or more aspects has been presented for purposes of illustration and description. It is not intended to be exhaustive or limiting to the precise form disclosed. Modifications or variations are possible in light of the above teachings. The one or more aspects were chosen and described in order to illustrate principles and practical application to thereby enable one of ordinary skill in the art to utilize the various aspects and with various modifications as are suited to the particular use contemplated. It is intended that the claims submitted herewith define the overall scope.

1. An end effector, comprising:
a first jaw member comprising a first electrode, the first jaw member comprising a distal end and a proximal end;
a second jaw member comprising a second electrode, wherein either the first or second jaw member is movable relative to the other between open and closed positions, the second jaw member comprising a distal end and a proximal end;
an electrically conductive member located at the distal end of either the first jaw member or the second jaw member, the electrically conductive member sized and configured to define a minimum distance between the first and second electrodes, the electrically conductive member having a first stiffness; and
an electrically insulative member located either on the first jaw member or the second jaw member, the electrically insulative member sized and configured to engage tissue, the electrically insulative member having a second stiffness, wherein the first stiffness is greater than the second stiffness.

2. The end effector of claim 1, wherein the electrically insulative member is configured to engage tissue located between the first and second jaw members.

3. The end effector of claim 1, wherein the electrically conductive member is configured to define a uniform distance between the first and second jaw members along the length of the first and second electrodes.

4. The end effector of claim 1, wherein the electrically conductive member is configured to define a non-uniform distance between the first and second jaw members.

5. The end effector of claim 1, wherein the electrically insulative member comprises two or more electrically insulative members.

6. The end effector of claim 5, wherein the two or more electrically insulative members have different stiffness, which is less than the first stiffness of the electrically conductive member.

7. The end effector of claim 5, wherein each of the two or more electrically insulative members are configured to deform to different extents.

8. The end effector of claim 5, wherein each of the two or more electrically insulative members are configured to deform uniformly.

9. The end effector of claim 1, comprising an embossed member disposed on either the first or second electrode, wherein the electrically insulative member is provided on the embossed member.

10. The end effector of claim 8, wherein the embossed member is located in an inner portion of the first jaw member.

11. The end effector of claim 1, wherein either the first or second jaw member comprises a cambered electrode.

12. The end effector of claim 10, wherein the first and second jaw members are in the closed position and the electrically insulative member located on the first jaw member does not contact the second electrode.

13. The end effector of claim 12, wherein the first and second jaw members are in the closed position and the electrically insulative member located on the second jaw member does not contact the first electrode.

14. The end effector of claim 1, wherein one of the first or second jaw members is configured to deform.

15. The end effector of claim 1, wherein one of the first or second jaw members comprises a stepped electrode defining different planes.

16. The end effector of claim 1, wherein one of first or second jaw members define at least one recess corresponding to the electrically insulative member.

17. The end effector of claim 1, wherein one of first or second jaw members define at least one recess corresponding to the electrically conductive member.

18. An electrosurgical device, comprising:
a handle assembly;
an end effector operably coupled to the handle assembly the end effector, comprising:
a first jaw member comprising a first electrode, the first jaw member comprising a distal end and a proximal end;
a second jaw member comprising a second electrode, wherein either the first or second jaw member is movable relative to the other between open and closed positions, the second jaw member comprising a distal end and a proximal end;
an electrically conductive member located at the distal end of the first jaw member, the electrically conductive member sized and configured to define a minimum distance, the electrically conductive member having a first stiffness; and
an electrically insulative member located either on the first jaw member or the second jaw member, the electrically insulative member sized and configured to engage tissue, the electrically insulative member having a second stiffness, wherein the first stiffness is greater than the second stiffness; and
a connecting member configured to connect the handle assembly and the end effector.

19. An electrosurgical system, comprising:
an electrosurgical energy generator;
an electrosurgical device comprising a handle assembly, an end effector operably coupled to the handle assembly, and a connecting member configured to connect the handle assembly and the end effector, the end effector comprising:
a first jaw member comprising a first electrode, the first electrode comprising a distal end and a proximal end;
a second jaw member comprising a second electrode, wherein either the first or second jaw member is movable relative to the other between open and closed positions, the second jaw member comprising a distal end and a proximal end;
an electrically conductive member located at the distal end of the first jaw member, the electrically conductive member sized and configured to define a minimum distance between the first and second electrodes, the electrically conductive member having a first stiffness; and
an electrically insulative member located either on the first jaw member or the second jaw member, the electrically insulative member sized and configured to engage tissue, the electrically insulative member having a second stiffness, wherein the first stiffness is greater than the second stiffness.

20. A method of manufacturing an end effector comprising a first jaw member, a second jaw member, and an electrically insulative member assembly, the method comprising:
providing a position setting material in an inner portion of the first jaw member;
inserting into the inner portion of the first jaw member the electrically insulative member assembly; and
providing a position setting member between the first and second jaw members, the position setting member having non-uniform thickness.

21. The method of claim 20, further comprising:
setting the position setting material; and
removing the position setting member.
